(19) United States
(12) Patent Application Publication
(10) Pub. No.: US 2003/0031584 A1
(43) Pub. Date: Feb. 13, 2003

(54) Methods for sterilizing biological materials using dipeptide stabilizers

(76) Inventors: Wilson Burgess, Great Oak Lane, CA (US); William N. Drohan, Springfield, VA (US); Martin J. MacPhee, Montgomery Village, MD (US); David M. Mann, Gaithersburg, MD (US); Ewa Maddox, Rockville, MD (US)

Correspondence Address: FLESHNER & KIM, LLP, P.O. Box 221200, Chantilly, VA, 20153-1200, US

(21) Appl. No.: 09/925,619

(22) Filed: Aug. 10, 2001

(51) Int. Cl. A61L 2/08

(52) U.S. Cl. 422/22

(57) ABSTRACT

Methods are disclosed for sterilizing biological materials to reduce the level of active biological contaminants or pathogens such as viruses, bacteria, nanobacteria, yeasts, molds, mycoplasmas, ureaplasmas, prions and parasites. These methods involve the use of dipeptide stabilizers in methods of sterilizing biological materials with irradiation.

### FIELD OF THE INVENTION

The present invention relates to methods for sterilizing biological materials to reduce the level of one or more biological contaminants or pathogens therein, such as viruses, bacteria, nanobacteria, yeasts, molds, mycoplasmas, ureaplasmas, prions and/or parasites. The present invention particularly relates to the use of dipeptide stabilizers in methods of sterilizing biological materials with irradiation.

### BACKGROUND OF THE INVENTION

Many biological materials that are prepared for human, veterinary, diagnostic and/or experimental use may contain unwanted and potentially dangerous biological contaminants or pathogens, such as viruses, bacteria, nanobacteria, yeasts, molds, mycoplasmas, ureaplasmas, prions and parasites. Consequently, it is of utmost importance that any biological contaminant in the biological material be inactivated before the product is used. This is especially critical when the material is to be administered directly to a patient, for example in blood transfusions, blood factor replacement therapy, organ transplants and other forms of human therapy corrected or treated by intravenous, intramuscular or other forms of injection. This is also critical for the various biological materials that are prepared in media or via culture of cells or recombinant cells which contain various types of plasma and/or plasma derivatives or other biologic materials and which may be subject to mycoplasma, prion, bacterial and/or viral contaminants.

Most procedures for producing biological materials have involved methods that screen or test the biological materials for one or more particular biological contaminants or pathogens rather than removal or inactivation of the contaminant(s) and/or pathogen(s) from the material. Materials that test positive for a biological contaminant or pathogen are merely not used. Examples of screening procedures include the testing for a particular virus in human blood from blood donors. Such procedures, however, are not always reliable and are not able to detect the presence of certain viruses, particularly in very low numbers. This reduces the value or certainty of the test in view of the consequences associated with a false negative result. False negative results can be life threatening in certain cases, for example in the case of Acquired Immune Deficiency Syndrome (AIDS). Furthermore, in some instances it can take weeks, if not months, to determine whether or not the material is contaminated. Therefore, it would be desirable to apply techniques that would kill or inactivate biological contaminants and pathogens during and/or after manufacturing the biological material.

In conducting experiments to determine the ability of technologies to inactivate viruses, the actual viruses of concern are seldom utilized. This is a result of safety concerns for the workers conducting the tests, and the difficulty and expense associated with the containment facilities and waste disposal. In their place, model viruses of the same family and class are used.

In general, it is acknowledged that the most difficult viruses to inactivate are those with an outer shell made up of proteins, and that among these, the most difficult to inactivate are those of the smallest size. This has been shown to be true for gamma irradiation and most other forms of radiation as these viruses' diminutive size is associated with a small genome. The magnitude of direct effects of radiation upon a molecule are directly proportional to the size of the molecule, that is the larger the target molecule, the greater the effect. As a corollary, it has been shown for gamma-irradiation that the smaller the viral genome, the higher the radiation dose required to inactive it.

Among the viruses of concern for both human and animal-derived biological materials, the smallest, and thus most difficult to inactivate, belong to the family of Parvoviruses and the slightly larger protein-coated Hepatitis virus. In humans, the Parvovirus B19, and Hepatitis A are the agents of concern. In porcine-derived materials, the smallest corresponding virus is Porcine Parvovirus. Since this virus is harmless to humans, it is frequently chosen as a model virus for the human B19 Parvovirus. The demonstration of inactivation of this model parvovirus is considered adequate proof that the method employed will kill human B19 virus and Hepatitis A, and by extension, that it will also kill the larger and less hardy viruses such as HIV, CMV, Hepatitis B and C and others.

More recent efforts have focussed on methods to remove or inactivate contaminants in the products. Such methods include heat treating, filtration and the addition of chemical inactivants or sensitizers to the product.

Heat treatment requires that the product be heated to approximately 60° C. for about 70 hours which can be damaging to sensitive products. In some instances, heat inactivation can actually destroy 50% or more of the biological activity of the product.

Filtration involves filtering the product in order to physically remove contaminants. Unfortunately, this method may also remove products that have a high molecular weight. Further, in certain cases, small viruses may not be removed by the filter.

The procedure of chemical sensitization involves the addition of noxious agents which bind to the DNA/RNA of the virus and which are activated either by UV or other radiation. This radiation produces reactive intermediates and/or free radicals which bind to the DNA/RNA of the virus, break the chemical bonds in the backbone of the DNA/RNA, and/or cross-link or complex it in such a way that the virus can no longer replicate. This procedure requires that unbound sensitizer is washed from products since the sensitizers are toxic, if not mutagenic or carcinogenic, and cannot be administered to a patient.

Irradiating a product with gamma radiation is another method of sterilizing a product. Gamma radiation is effective in destroying viruses and bacteria when given in high total doses (Keathly et al., “Is There Life After Irradiation? Part 2,” BioPharm July-August, 1993, and Leitman, Use of Blood Cell Irradiation in the Prevention of Post Transfusion Graft-vs-Host Disease,” Transfusion Science 10:219-239 (1989)). The published literature in this area, however, teaches that gamma radiation can be damaging to radiation sensitive products, such as blood, blood products, protein and protein-containing products. In particular, it has been shown that high radiation doses are injurious to red cells, platelets and granulocytes (Leitman). U.S. Pat. No. 4,620,908 discloses that protein products must be frozen prior to irradiation in order to maintain the viability of the protein product. This patent concludes that “[i]f the gamma irradiation were applied while the protein material was at, for example, ambient temperature, the material would be also completely destroyed, that is the activity of the material would be rendered so low as to be virtually ineffective”. Unfortunately, many sensitive biological materials, such as monoclonal antibodies (Mab), may lose viability and activity if subjected to freezing for irradiation purposes and then thawing prior to administration to a patient.

In view of the difficulties discussed above, there remains a need for methods of sterilizing compositions containing one or more biological materials that are effective for reducing the level of active biological contaminants or pathogens without an adverse effect on the material(s).

### SUMMARY OF THE INVENTION

Accordingly, it is an object of the present invention to provide methods of sterilizing biological compositions by reducing the level of active biological contaminants or pathogens without adversely effecting the composition. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof.

In accordance with these and other objects, a first embodiment of the present invention is directed to a method for sterilizing a biological material that is sensitive to radiation comprising: (i) adding to a biological material at least one dipeptide stabilizer in an amount effective to protect the biological material from radiation; and (ii) irradiating the biological material with radiation at an effective rate for a time effective to sterilize the biological material

Another embodiment of the present invention is directed to a method for sterilizing a biological material that is sensitive to radiation comprising: (i) reducing the residual solvent content of a biological material; (ii) adding to the biological material at least one dipeptide stabilizer; and (iii) irradiating the biological material with radiation at an effective rate for a time effective to sterilize the biological material, wherein the level of residual solvent content and the amount of dipeptide stabilizer are together effective to protect the biological material from radiation. According to this embodiment, steps (i) and (ii) may be reversed.

Another embodiment of the present invention is directed to a method for sterilizing a biological material that is sensitive to radiation comprising: (i) reducing the temperature of a biological material; (ii) adding to the biological material at least one dipeptide stabilizer; and (iii) irradiating the biological material with radiation at an effective rate for a time effective to sterilize the biological material, wherein the temperature and the amount of dipeptide stabilizer are together effective to protect the biological material from radiation. According to this embodiment, steps (i) and (ii) may be reversed.

The invention also provides a biological composition comprising at least one biological material and a least one dipeptide stabilizer in an amount effective to preserve said biological material for its intended use following sterilization with radiation.

### BRIEF DESCRIPTION OF THE DRAWINGS

FIGS. 1A-1C shows the protective effect of the stabilizers on gamma irradiated immunoglobulin preparations.

FIGS. 2A-2E show the protective effect of stabilizers on immunoglobulin preparations.

FIGS. 3A-3H show the protective effect of ascorbate, alone or in combination with Gly—Gly, on a liquid polyclonal antibody preparation.

FIGS. 4A-4C show the protective effect of stabilizers on lyophilized anti-insulin monoclonal immunoglobulin irradiated at a high dose rate.

FIGS. 5A-5B show the protective effect of stabilizers on liquid anti-insulin monoclonal immunoglobulin irradiated to 45 kGy.

FIGS. 6A-6B show the protective effect of stabilizers on two different frozen enzyme preparations (a glycosidase and a sulfatase).

FIG. 7 shows the protective effect of ascorbate (200 mM) and a combination of ascorbate (200 mM) and Gly—Gly (200 mM) on a frozen glycosidase preparation.

FIG. 8 protective effect of various stabilizers on anti-insulin monoclonal immunoglobulin supplemented with 0.1% human serum albumin (HSA) exposed to gamma irradiation up to 100 kGy.

FIG. 9 shows the protective effect of the dipeptide stabilizer L-carnosine, alone or in combination with ascorbate, on gamma irradiated liquid urokinase.

FIG. 10 shows the protective effect of the dipeptide stabilizer anserine on gamma irradiated liquid urokinase.

FIG. 11 shows the protective effect of L-carnosine on gamma irradiated liquid urokinase.

FIG. 12 shows the protective effect of L-camosine on gamma irradiated immobilized anti-insulin monoclonal immunoglobulin.

FIG. 13 shows the protective effect of L-carnosine, alone or in combination with ascorbate, on gamma irradiated immobilized anti-insulin monoclonal immunoglobulin.

FIG. 14 shows the protective effect of L-camosine, alone or in combination with ascorbate, on gamma irradiated lyophilized Factor VIII.

### DETAILED DESCRIPTION OF THE PREFERRED EMBODIMENTS

A. Definitions

Unless defined otherwise, all technical and scientific terms used herein are intended to have the same meaning as is commonly understood by one of ordinary skill in the relevant art.

As used herein, the singular forms “a,” “an,” and “the” include the plural reference unless the context clearly dictates otherwise.

As used herein, the term “biological material” is intended to mean any substance derived or obtained from a living organism. Illustrative examples of biological materials include, but are not limited to, the following: cells; tissues; blood or blood components; proteins, including recombinant and transgenic proteins, and proetinaceous materials; enzymes, including digestive enzymes, such as trypsin, chymotrypsin, alpha-glucosidase and iduronodate-2-sulfatase; immunoglobulins, including mono and polyimmunoglobulins; botanicals; food and the like. Preferred examples of biological materials include, but are not limited to, the following: ligaments; tendons; nerves; bone, including demineralized bone matrix, grafts, joints, femurs, femoral heads, etc.; teeth; skin grafts; bone marrow, including bone marrow cell suspensions, whole or processed; heart valves; cartilage; corneas; arteries and veins; organs, including organs for transplantation, such as hearts, livers, lungs, kidneys, intestines, pancreas, limbs and digits; lipids; carbohydrates; collagen, including native, afibrillar, atelomeric, soluble and insoluble, recombinant and transgenic, both native sequence and modified; chitin and its derivatives, including NO-carboxy chitosan (NOCC); stem cells, islet of Langerhans cells and other cells for transplantation, including genetically altered cells; red blood cells; white blood cells, including monocytes; and platelets.

As used herein, the term “sterilize” is intended to mean a reduction in the level of at least one active or potentially active biological contaminant or pathogen found in the biological material being treated according to the present invention.

As used herein, the term “biological contaminant or pathogen” is intended to mean a contaminant or pathogen that, upon direct or indirect contact with a biological material, may have a deleterious effect on a biological material or upon a recipient thereof. Such biological contaminants or pathogens include the various viruses, prions, molds, yeasts, bacteria, nanobacteria, mycoplasmas, ureaplasmas and parasites known to those of skill in the art to generally be found in or infect biological materials. Examples of biological contaminants or pathogens include, but are not limited to, the following: viruses, such as human immunodeficiency viruses and other retroviruses, herpes viruses, filoviruses, circoviruses, paramyxoviruses, cytomegaloviruses, hepatitis viruses (including hepatitis A, B and C and variants thereof), pox viruses, toga viruses, Ebstein-Barr viruses and parvoviruses; bacteria, such as Escherichia, Bacillus, Campylobacter, Streptococcus and Staphalococcus; nanobacteria; parasites, such as Trypanosoma and malarial parasites, including Plasmodium species; yeasts; molds; mycoplasmas; ureaplasmas; and prions responsible for TSE (transmissible spongiform encephalopathies), such as scrapie, kuru, BSE (bovine spongiform encephalopathy), CJD (Creutzfeldt-Jakob disease), Gerstmann-Straeussler-Scheinkler syndrome and fatal familial insomnia. As used herein, the term “active biological contaminant or pathogen” is intended to mean a biological contaminant or pathogen that is capable of causing a deleterious effect, either alone or in combination with another factor, such as a second biological contaminant or pathogen or a native protein (wild-type or mutant) or antibody, in the biological material and/or a recipient thereof.

As used herein, the term “blood components” is intended to mean one or more of the components that may be separated from whole blood and include, but are not limited to, the following: cellular blood components, such as red blood cells, white blood cells and platelets; blood proteins, such as blood clotting factors, enzymes, albumin, plasminogen, fibrinogen and immunoglobulins; and liquid blood components, such as plasma, plasma protein fraction (PPF), cryoprecipitate, plasma fractions and plasma-containing compositions.

As used herein, the term “cellular blood component” is intended to mean one or more of the components of whole blood that comprises cells, such as red blood cells, white blood cells, stem cells and platelets.

As used herein, the term “blood protein” is intended to mean one or more of the proteins that are normally found in whole blood. Illustrative examples of blood proteins found in mammals, including humans, include, but are not limited to, the following: coagulation proteins, both vitamin K-dependent, such as Factor VII and Factor IX, and non-vitamin K-dependent, such as Factor VIII and von Willebrands factor; albumin; lipoproteins, including high density lipoproteins and low density lipoproteins; complement proteins; globulins, such as immunoglobulins IgA, IgM, IgG and IgE; and the like. A preferred group of blood proteins includes Factor I (fibrinogen), Factor II (prothrombin), Factor III (tissue factor), Factor V (proaccelerin), Factor VI (accelerin), Factor VII (proconvertin, serum prothrombin conversion), Factor VIII (antihemophiliac factor A), Factor IX (antihemophiliac factor B), Factor X (Stuart-Prower factor), Factor XI (plasma thromboplastin antecedent), Factor XII (Hageman factor), Factor XIII (protransglutamidase), von Willebrands factor (vWF), Factor Ia, Factor Ia, Factor IlIa, Factor Va, Factor VIa, Factor VIIa, Factor Villa, Factor IXa, Factor Xa, Factor XIa, Factor XIIa and Factor XIIIa. Another preferred group of blood proteins includes proteins found inside red blood cells, such as hemoglobin and various growth factors, and derivatives of these proteins.

As used herein, the term “liquid blood component” is intended to mean one or more of the fluid, non-cellular components of whole blood, such as plasma (the fluid, non-cellular portion of the whole blood of humans or animals as found prior to coagulation) and serum (the fluid, non-cellular portion of the whole blood of humans or animals as found after coagulation).

As used herein, the term “a biologically compatible solution” is intended to mean a solution to which a biological material may be exposed, such as by being suspended or dissolved therein, and remain viable, i.e., retain its essential biological and physiological characteristics.

As used herein, the term “a biologically compatible buffered solution” is intended to mean a biologically compatible solution having a pH and osmotic properties (e.g, tonicity, osmolality and/or oncotic pressure) suitable for maintaining the integrity of the material(s) therein. Suitable biologically compatible buffered solutions typically have a pH between 4 and 8.5 and are isotonic or only moderately hypotonic or hypertonic. Biologically compatible buffered solutions are known and readily available to those of skill in the art.

As used herein, the term “dipeptide stabilizer” is intended to mean a compound composed of two amino acids that reduces any damage to the biological material being irradiated to a level that is insufficient to preclude the safe and effective use of the biological material. The amino acids may be either naturally occurring amino acids, i.e. L-amino acids, or non-naturally occurring amino acids, i.e., D-amino acids. Derivatives and analogs of amino acids may also be employed. Preferred amino acids include, but are not limited to, the following: glycine; histidine; glutamic acid; tryptophan; cysteine; and methionine; and derivatives thereof, such as N-acetylcysteine (NAC) and sodium capryl N-acetyl tryptophan. Preferred dipeptide stabilizers are homologous dipeptide stabilizers, i.e., dipeptide stabilizers composed of two identical amino acids. Particularly preferred homologous dipeptide stabilizers are composed of naturally occurring amino acids, such as Gly—Gly (glycylglycine) and Trp-Trp. Other preferred dipeptide stabilizers are heterologous dipeptide stabilizers, i.e., dipeptide stabilizers composed of different amino acids, such as camosine (β-alanyl-histidine), anserine (β-alanyl-methylhistidine) and Gly—Trp.

As used herein, the term “additional stabilizer” is intended to mean a compound or material that, alone and/or in combination with at least one dipeptide stabilizer, reduces damage to the biological material being irradiated to a level that is insufficient to preclude the safe and effective use of the material. Illustrative examples of additional stabilizers include, but are not limited to, the following: antioxidants; free radical scavengers, including spin traps; combination stabilizers, i.e. stabilizers which are effective at quenching both Type I and Type II photodynamic reactions; and ligands, such as heparin, that stabilize the molecules to which they bind. Preferred examples of additional stabilizers include, but are not limited to, the following: fatty acids, including 6,8-dimercapto-octanoic acid (lipoic acid) and its derivatives and analogues (alpha, beta, dihydro, bisno and tetranor lipoic acid), thioctic acid, 6,8-dimercapto-octanoic acid, dihydrolopoate (DL-6,8-dithioloctanoic acid methyl ester), lipoamide, bisonor methyl ester and tatranor-dihydrolipoic acid, furan fatty acids, oleic and linoleic and palmitic acids and their salts and derivatives; flavonoids, phenylpropaniods, and flavenols, such as quercetin, rutin and its derivatives, apigenin, aminoflavone, catechin, hesperidin and, naringin; carotenes, including beta-carotene; Co-Q10; xanthophylls; polyhydric alcohols, such as glycerol, mannitol; sugars, such as xylose, glucose, ribose, mannose, fructose and trehalose; amino acids and derivatives thereof, such as histidine, N-acetylcysteine (NAC), glutamic acid, tryptophan, sodium capryl N-acetyl tryptophan and methionine; azides, such as sodium azide; enzymes, such as Superoxide Dismutase (SOD) and Catalase; uric acid and its derivatives, such as 1,3-dimethyluric acid and dimethylthiourea; allopurinol; thiols, such as glutathione and reduced glutathione and cysteine; trace elements, such as selenium; vitamins, such as vitamin A, vitamin C (including its derivatives and salts such as sodium ascorbate and palmitoyl ascorbic acid) and vitamin E (and its derivatives and salts such as tocopherol acetate and alpha-tocotrienol); chromanol-alpha-C6; 6-hydroxy-2,5,7,8-tetramethylchroma-2 carboxylic acid (Trolox) and derivatives; extraneous proteins, such as gelatin and albumin; tris-3-methyl-1-phenyl-2-pyrazolin-5-one (MCI-186); citiolone; puercetin; chrysin; dimethyl sulfoxide (DMSO); piperazine diethanesulfonic acid (PIPES); imidazole; methoxypsoralen (MOPS); 1,2-dithiane-4,5-diol; reducing substances, such as butylated hydroxyanisole (BHA) and butylated hydroxytoluene (BHT); cholesterol; probucol; indole derivatives; thimerosal; lazaroid and tirilazad mesylate; proanthenols; proanthocyanidins; ammonium sulfate; Pegorgotein (PEG-SOD); N-tert-butyl-alpha-phenylnitrone (PBN); 4-hydroxy-2,2,6,6-tetramethylpiperidin-1-oxyl (Tempol); mixtures of ascorbate, urate and Trolox C (Asc/urate/Trolox C); proteins and peptides of at least three amino acids, in which each amino acid may be in its D or L form; diosmin; pupurogalin; gallic acid and its derivatives including but not limited to propyl gallate, sodium formaldehyde sulfoxylate and silymarin. Particularly preferred examples include single stabilizers or combinations of stabilizers that are effective at quenching both Type I and Type II photodynamic reactions and volatile stabilizers, which can be applied as a gas and/or easily removed by evaporation, low pressure and similar methods.

As used herein, the term “residual solvent content” is intended to mean the amount or proportion of freely-available liquid in the biological material. Freely-available liquid means the liquid, such as water or an organic solvent (e.g. ethanol, isopropanol, polyethylene glycol, etc.), present in the biological material being sterilized that is not bound to or complexed with one or more of the non-liquid components of the biological material. Freely-available liquid includes intracellular water. The residual solvent contents related as water referenced herein refer to levels determined by the FDA approved, modified Karl Fischer method (Meyer and Boyd, Analytical Chem., 31:215-219, May, 1959; et al., J. Biol. Standardization, 10:249-259, 1982; Centers for Biologics Evaluation and Research, FDA, Docket No. 89D-0140, 83-93; 1990) or by near infrared spectroscopy. Quantitation of the residual levels of other solvents may be determined by means well known in the art, depending upon which solvent is employed. The proportion of residual solvent to solute may also be considered to be a reflection of the concentration of the solute within the solvent. When so expressed, the greater the concentration of the solute, the lower the amount of residual solvent.

As used herein, the term “sensitizer” is intended to mean a substance that selectively targets viral, bacterial, prion and/or parasitic contaminants, rendering them more sensitive to inactivation by radiation, therefore permitting the use of a lower rate or dose of radiation and/or a shorter time of irradiation than in the absence of the sensitizer. Illustrative examples of suitable sensitizers include, but are not limited to, the following: psoralen and its derivatives and analogs (including 3-carboethoxy psoralens); inactines and their derivatives and analogs; angelicins, khellins and coumarins which contain a halogen substituent and a water solubilization moiety, such as quaternary ammonium ion or phosphonium ion; nucleic acid binding compounds; brominated hematoporphyrin; phthalocyanines; purpurins; porphorins; halogenated or metal atom-substituted derivatives of dihematoporphyrin esters, hematoporphyrin derivatives, benzoporphyrin derivatives, hydrodibenzoporphyrin dimaleimade, hydrodibenzoporphyrin, dicyano disulfone, tetracarbethoxy hydrodibenzoporphyrin, and tetracarbethoxy hydrodibenzoporphyrin dipropionamide; doxorubicin and daunomycin, which may be modified with halogens or metal atoms; netropsin; BD peptide, S2 peptide; S-303 (ALE compound); dyes, such as hypericin, methylene blue, eosin, fluoresceins (and their derivatives), flavins, merocyanine 540; photoactive compounds, such as bergapten; and SE peptide.

As used herein, the term “proteinaceous material” is intended to mean any material derived or obtained from a living organism that comprises at least one protein or peptide. A proteinaceous material may be a naturally occurring material, either in its native state or following processing/purification and/or derivatization, or an artificially produced material, produced by chemical synthesis or recombinant/transgenic technology and, optionally, process/purified and/or derivatized. Illustrative examples of proteinaceous materials include, but are not limited to, the following: proteins and peptides produced from cell culture; milk and other dairy products; ascites; hormones; growth factors; materials, including pharmaceuticals, extracted or isolated from animal tissue, such as heparin and insulin, or plant matter; plasma, including fresh, frozen and freeze-dried, and plasma protein fraction; fibrinogen and derivatives thereof, fibrin, fibrin I, fibrin II, soluble fibrin and fibrin monomer, and/or fibrin sealant products; whole blood; protein C; protein S; alpha-i anti-trypsin (alpha-I protease inhibitor); butyl-cholinesterase; anticoagulants, such as coumarin drugs (warfarin); streptokinase; tissue plasminogen activator (tPA); erythropoietin (EPO); urokinase; neupogen; anti-thrombin-3; alpha-glucosidase; (fetal) bovine serum/horse serum; meat; immunoglobulins, including anti-sera, monoclonal antibodies, polyclonal antibodies and genetically engineered or produced antibodies; albumin; alpha-globulins; beta-globulins; gamma-globulins; coagulation proteins; complement proteins; and interferons.

As used herein, the term “radiation” is intended to mean radiation of sufficient energy to sterilize at least some component of the irradiated biological material. Types of radiation include, but are not limited to, the following: (i) corpuscular (streams of subatomic particles such as neutrons, electrons, and/or protons); (ii) electromagnetic (originating in a varying electromagnetic field, such as radio waves, visible (both mono and polychromatic) and invisible light, infrared, ultraviolet radiation, x-radiation, and gamma rays and mixtures thereof); and (iii) sound and pressure waves. Such radiation is often described as either ionizing (capable of producing ions in irradiated materials) radiation, such as gamma rays, and non-ionizing radiation, such as visible light. The sources of such radiation may vary and, in general, the selection of a specific source of radiation is not critical provided that sufficient radiation is given in an appropriate time and at an appropriate rate to effect sterilization. In practice, gamma radiation is usually produced by isotopes of Cobalt or Cesium, while UV and X-rays are produced by machines that emit UV and X-radiation, respectively, and electrons are often used to sterilize materials in a method known as “e-beam” irradiation that involves their production via a machine. Visible light, both mono- and polychromatic, is produced by machines and may, in practice, be combined with invisible light, such as infrared and UV, that is produced by the same machine or a different machine.

As used herein, the term “to protect” is intended to mean to reduce any damage to the biological material being irradiated, that would otherwise result from the irradiation of that material, to a level that is insufficient to preclude the safe and effective use of the material following irradiation. In other words, a substance or process “protects” a biological material from radiation if the presence of that substance or carrying out that process results in less damage to the material from irradiation than in the absence of that substance or process. Thus, biological material may be used safely and effectively after irradiation in the presence of a substance or following performance of a process that “protects” the material, but could not be used safely and effectively after irradiation under identical conditions but in the absence of that substance or the performance of that process.

B. Particularly Preferred Embodiments

A first preferred embodiment of the present invention is directed to a method for sterilizing a biological material that is sensitive to radiation comprising: (i) adding to a biological material at least one dipeptide stabilizer in an amount effective to protect the biological material from radiation; and (ii) irradiating the biological material with radiation at an effective rate for a time effective to sterilize the biological material.

A second preferred embodiment of the present invention is directed to a method for sterilizing a biological material that is sensitive to radiation comprising: (i) reducing the residual solvent content of a biological material; (ii) adding to the biological material at least one dipeptide stabilizer; and (iii) irradiating the biological material with radiation at an effective rate for a time effective to sterilize the biological material, wherein the level of residual solvent content and the amount of dipeptide stabilizer are together effective to protect the biological material from radiation. The order of steps (i) and (ii) may, of course, be reversed as desired.

A third preferred embodiment of the present invention is directed to a method for sterilizing a biological material that is sensitive to radiation comprising: (i) reducing the temperature of a biological material; (ii) adding to the biological material at least one dipeptide stabilizer; and (iii) irradiating the biological material with radiation at an effective rate for a time effective to sterilize the biological material, wherein the temperature and the amount of dipeptide stabilizer are together effective to protect the biological material from radiation. The order of steps (i) and (ii) may, of course, be reversed as desired.

According to the methods of the present invention, a dipeptide stabilizer is added prior to irradiation of the biological material with radiation. This dipeptide stabilizer is preferably added to the biological material in an amount that is effective to protect the biological material from the radiation. Suitable amounts of dipeptide stabilizer may vary depending upon certain features of the particular method(s) of the present invention being employed, such as the particular dipeptide stabilizer being used and/or the nature and characteristics of the particular biological material being irradiated and/or its intended use, and can be determined empirically by one skilled in the art.

According to certain methods of the present invention, an additional stabilizer is added to the biological material prior to irradiation of the biological material with radiation. This additional stabilizer is preferably added in an amount that is effective in combination with the dipeptide stabilizer to protect the biological material from the radiation. Suitable amounts of additional stabilizer may vary depending upon certain features of the particular method(s) of the present invention being employed, such as the particular stabilizer(s) being used and/or the nature and characteristics of the particular biological material being irradiated and/or its intended use, and can be determined empirically by one skilled in the art.

According to certain methods of the present invention, the residual solvent content of the biological material is reduced prior to irradiation of the biological material with radiation. The residual solvent content is preferably reduced to a level that is effective to protect the biological material from the radiation, either alone or in combination with the dipeptide stabilizer and any additional stabilizer. Suitable levels of residual solvent content may vary depending upon certain features of the particular method(s) of the present invention being employed, such as the particular dipeptide stabilizer being used and/or the nature and characteristics of the particular biological material being irradiated and/or its intended use, and can be determined empirically by one skilled in the art. There may be biological materials for which it is desirable to maintain the residual solvent content to within a particular range, rather than a specific value.

When the solvent is water, and particularly when the biological material is in a solid phase, the residual solvent content is generally less than about 15%, typically less than about 10%, usually less than about 5%, preferably less than about 3.0%, more preferably less than about 2.0%, even more preferably less than about 1.0%, still more preferably less than about 0.5%, still even more preferably less than about 0.2% and most preferably less than about 0.08%.

The solvent may preferably be a non-aqueous solvent, more preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile solvents are particularly preferred.

In a preferred embodiment, when the residual solvent is water, the residual solvent content of a biological material is reduced by dissolving or suspending the biological material in a non-aqueous solvent that is capable of dissolving water. Preferably, such a non-aqueous solvent is not prone to the formation of free-radicals upon irradiation and has little or no dissolved oxygen or other gas(es) that is (are) prone to the formation of free-radicals upon irradiation.

When the biological material is in a liquid phase, reducing the residual solvent content may be accomplished by any of a number of means, such as by increasing the solute concentration. In this manner, the concentration of the biological material dissolved within the solvent may be increased to generally at least about 0.5%, typically at least about 1%, usually at least about 5%, preferably at least about 10%, more preferably at least about 15%, even more preferably at least about 20%, still even more preferably at least about 25%, and most preferably at least about 50%.

In certain embodiments of the present invention, the residual solvent content of a particular biological material may be found to lie within a range, rather than at a specific point. Such a range for the preferred residual solvent content of a particular biological material may be determined empirically by one skilled in the art.

While not wishing to be bound by any theory of operability, it is believed that the reduction in residual solvent content reduces the degrees of freedom of the biological material, reduces the number of targets for free radical generation and may restrict the solubility of these free radicals. Similar results might therefore be achieved by lowering the temperature of the biological material below its eutectic point or below its freezing point, or by vitrification to likewise reduce the degrees of freedom of the biological material. These results may permit the use of a higher rate and/or dose of radiation than might otherwise be acceptable. Thus, the methods described herein may be performed at any temperature that doesn't result in unacceptable damage to the biological material, i.e., damage that would preclude the safe and effective use of the biological material. Preferably, the methods described herein are performed at ambient temperature or below ambient temperature, such as below the eutectic point or freezing point of the biological material being irradiated.

In accordance with the methods of the present invention, an “acceptable level” of damage may vary depending upon certain features of the particular method(s) of the present invention being employed, such as the nature and characteristics of the particular biological material and/or dipeptide stabilizer being used, and/or the intended use of the biological material being irradiated, and can be determined empirically by one skilled in the art. An “unacceptable level” of damage would therefore be a level of damage that would preclude the safe and effective use of the biological material being sterilized. The particular level of damage in a given biological material may be determined using any of the methods and techniques known to one skilled in the art.

The residual solvent content of the biological material may be reduced by any of the methods and techniques known to those skilled in the art for reducing solvent from a biological material without producing an unacceptable level of damage to the biological material. Such methods include, but are not limited to, evaporation, concentration, centrifugal concentration, vitrification and spray-drying.

A particularly preferred method for reducing the residual solvent content of a biological material is lyophilization.

Another particularly preferred method for reducing the residual solvent content of a biological material is vitrification, which may be accomplished by any of the methods and techniques known to those skilled in the art, including the addition of solute and or additional solutes, such as sucrose, to raise the eutectic point of the biological material, followed by a a gradual application of reduced pressure to the biological material in order to remove the residual solvent, such as water. The resulting glassy material will then have a reduced residual solvent content.

According to certain methods of the present invention, the biological material to be sterilized may be immobilized upon a solid surface by any means known and available to one skilled in the art. For example, the biological material to be sterilized may be present as a coating or surface on a biological or non-biological substrate.

The radiation employed in the methods of the present invention may be any radiation effective for the inactivation of one or more active biological contaminants or pathogens in the biological material being treated. The radiation may be corpuscular, including e-beam radiation. Preferably the radiation is electromagnetic radiation, including x-rays, infrared, visible light, UV light and mixtures of various wavelengths of electromagnetic radiation. A particularly preferred form of radiation is gamma radiation.

According to the methods of the present invention, the biological material to be sterilized is irradiated with the radiation at a rate effective for the inactivation of one or more active biological contaminants or pathogens in the material, while not producing an unacceptable level of damage to that material. Suitable rates of irradiation may vary depending upon certain features of the methods of the present invention being employed, such as the nature and characteristics of the particular biological material being irradiated, the particular form of radiation involved and/or the particular biological contaminants or pathogens being inactivated. Suitable rates of irradiation can be determined empirically by one skilled in the art. Preferably, the rate of irradiation is constant for the duration of the sterilization procedure. When this is impractical or otherwise not desired, a variable or discontinuous irradiation may be utilized.

According to the methods of the present invention, the rate of irradiation may be optimized to produce the most advantageous combination of product recovery and time required to complete the operation. Both low (≦3 kGy/hour) and high (>3 kGy/hour) rates may be utilized in the methods described herein to achieve such results. The rate of irradiation is preferably be selected to optimize the recovery of the biological material while still sterilizing the biological material. Although reducing the rate of irradiation may serve to decrease damage to the biological material, it will also result in longer irradiation times being required to achieve a particular desired total dose. A higher dose rate may therefore be preferred in certain circumstances, such as to minimize logistical issues and costs, and may be possible when used in accordance with the methods described herein for protecting a biological material from irradiation.

According to a particularly preferred embodiment of the present invention, the rate of irradiation is not more than about 3.0 kGy/hour, more preferably between about 0.1 kGy/hr and 3.0 kGy/hr, even more preferably between about 0.25 kGy/hr and 2.0 kGy/hour, still even more preferably between about 0.5 kGy/hr and 1.5 kGy/hr and most preferably between about 0.5 kGy/hr and 1.0 kGy/hr.

According to another particularly preferred embodiment of the present invention, the rate of irradiation is at least about 3.0 kGy/hr, more preferably at least about 6 kGy/hr, even more preferably at least about 16 kGy/hr, and even more preferably at least about 30 kGy/hr and most preferably at least about 45 kGy/hr or greater.

According to the methods of the present invention, the biological material to be sterilized is irradiated with the radiation for a time effective for the inactivation of one or more active biological contaminants or pathogens in the biological material. Combined with irradiation rate, the appropriate irradiation time results in the appropriate dose of irradiation being applied to the biological material. Suitable irradiation times may vary depending upon the particular form and rate of radiation involved, the nature and characteristics of the particular biological material being irradiated and/or the particular biological contaminants or pathogens being inactivated. Suitable irradiation times can be determined empirically by one skilled in the art.

According to the methods of the present invention, the biological material to be sterilized is irradiated with radiation up to a total dose effective for the inactivation of one or more active biological contaminants or pathogens in the material, while not producing an unacceptable level of damage to that material. Suitable total doses of radiation may vary depending upon certain features of the methods of the present invention being employed, such as the nature and characteristics of the particular biological material being irradiated, the particular form of radiation involved and/or the particular biological contaminants or pathogens being inactivated. Suitable total doses of radiation can be determined empirically by one skilled in the art. Preferably, the total dose of radiation is at least 25 kGy, more preferably at least 45 kGy, even more preferably at least 75 kGy, and still more preferably at least 100 kGy or greater, such as 150 kGy or 200 kGy.

The particular geometry of the biological material being irradiated, such as the thickness and distance from the source of radiation, may be determined empirically by one skilled in the art.

According to certain methods of the present invention, an effective amount of at least one sensitizing compound may optionally be added to the biological material prior to irradiation, for example to enhance the effect of the irradiation on the biological contaminant(s) or pathogen(s) therein, while employing the methods described herein to minimize the deleterious effects of irradiation upon the biological material. Suitable sensitizers are known to those skilled in the art, and include psoralens and their derivatives and inactines and their derivatives.

According to the methods of the present invention, the irradiation of the biological material may occur at any temperature which is not deleterious to the biological material being sterilized. According to one preferred embodiment, the biological material is irradiated at ambient temperature. According to an alternate preferred embodiment, the biological material is irradiated at reduced temperature, i.e. a temperature below ambient temperature, such as 0° C., −40° C., −78° C. or −196° C. According to this embodiment of the present invention, the biological material is preferably irradiated at or below the freezing or eutectic point of the biological material. According to another alternate preferred embodiment, the biological material is irradiated at elevated temperature, i.e. a temperature above ambient temperature, such as 37° C., 60° C., 72° C. or 80° C. While not wishing to be bound by any theory, the use of elevated temperature may enhance the effect of irradiation on the biological contaminant(s) or pathogen(s) and therefore allow the use of a lower total dose of radiation.

According to the methods of the present invention, the irradiation of the biological material may occur at any pressure which is not deleterious to the biological material being sterilized. According to one preferred embodiment, the biological material is irradiated at elevated pressure. More preferably, the biological material is irradiated at elevated pressure due to the application of sound waves or the use of a volatile. While not wishing to be bound by any theory, the use of elevated pressure may enhance the effect of irradiation on the biological contaminant(s) or pathogen(s) and therefore allow the use of a lower total dose of radiation.

Generally, according to the methods of the present invention, the pH of the biological material undergoing sterilization is about 7. In some embodiments of the present invention, however, in order to avoid aggregation of the components of the biological material (such as in the case of immunoglobulins) or for other reasons, the biological material may have a pH of less than 7, preferably less than or equal to 6, more preferably less than or equal to 5, even more preferably less than or equal to 4, and most preferably less than or equal to 3. In alternative embodiments of the present invention, the biological material may have a pH of greater than 7, preferably greater than or equal to 8, more preferably greater than or equal to 9, even more preferably greater than or equal to 10, and most preferably greater than or equal to 11.

Similarly, according to the methods of the present invention, the irradiation of the biological material may occur under any atmosphere that is not deleterious to the biological material being treated. According to one preferred embodiment, the biological material is held in a low oxygen atmosphere or an inert atmosphere. When an inert atmosphere is employed, the atmosphere is preferably composed of a noble gas, such as helium or argon, more preferably a higher molecular weight noble gas, and most preferably argon. According to another preferred embodiment, the biological material is held under vacuum while being irradiated. According to a particularly preferred embodiment of the present invention, a biological material (lyophilized, liquid or frozen) is stored under vacuum or an inert atmosphere (preferably a noble gas, such as helium or argon, more preferably a higher molecular weight noble gas, and most preferably argon) prior to irradiation. According to an alternative preferred embodiment of the present invention, a liquid preparation of a biological material is held under low pressure, to decrease the amount of gas dissolved in the liquid, prior to irradiation, either with or without a prior step of solvent reduction, such as lyophilization.

In another preferred embodiment, where the biological material contains oxygen or other gases dissolved within or associated with it, the amount of these gases within or associated with the biological material may be reduced by any of the methods and techniques known and available to those skilled in the art, such as the controlled reduction of pressure within a container (rigid or flexible) holding the biological material to be treated or by placing the biological material in a container of approximately equal volume.

In certain embodiments of the present invention, when the biological material to be treated is a tissue, the dipeptide stabilizer is introduced according to any of the methods and techniques known and available to one skilled in the art, including soaking the tissue in a solution containing the dipeptide stabilizer, preferably under pressure, at elevated temperature and/or in the presence of a penetration enhancer, such as dimethylsulfoxide.

It will be appreciated that the combination of one or more of the features described herein may be employed to further minimize undesirable effects upon the biological material caused by irradiation, while maintaining adequate effectiveness of the irradiation process on the biological contaminant(s) or pathogen(s). For example, in addition to the use of a dipeptide stabilizer and an additional stabilizer, a particular biological material may also be lyophilized and kept under vacuum prior to irradiation to further minimize undesirable effects.

The sensitivity of a particular biological contaminant or pathogen to radiation is commonly calculated by determining the dose necessary to inactivate or kill all but 37% of the agent in a sample, which is known as the D₃₇ value. In accordance with certain preferred methods of the present invention, the sterilization of a biological material results in a decrease in the D₃₇ value of the biological contaminant or pathogen without a concomitant decrease in the D₃₇ value of the biological material. In accordance with other preferred methods of the present invention, the sterilization of a biological material results in an increase in the D₃₇ value of the biological material. In accordance with the most preferred methods of the present invention, the sterilization of a biological material results in a decrease in the D₃₇ value of the biological contaminant or pathogen and a concomitant increase in the D₃₇ value of the biological material.

### EXAMPLES

The following examples are illustrative, but not limiting, of the present invention. Other suitable modifications and adaptations are of the variety normally encountered by those skilled in the art and are fully within the spirit and scope of the present invention. Unless otherwise noted, all irradiation was accomplished using a ⁶⁰Co source.

### Example 1

In this experiment, the protective effect of the dipeptide Gly—Gly (20 mM) on gamma irradiated freeze-dried anti-insulin monoclonal immunoglobulin supplemented with 1% human serum albumin (HSA) and 5% sucrose was evaluated.

Methods

Samples were freeze-dried for approximately 64 hours and stoppered under vacuum and sealed with an aluminum, crimped seal. Samples were irradiated at a dose rate of 1.83-1.88 kGy/hr to a total dose of 45.1-46.2 kGy at 4° C.

Monoclonal immunoglobulin activity was determined by a standard ELISA protocol. Maxisorp plates were coated with human recombinant insulin at 2.5 μg/ml overnight at 4° C. The plate was blocked with 200 μl of blocking buffer (PBS, pH 7.4, 2% BSA) for two hours at 37° C. and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed six times with wash buffers. One hundred μl of Sigma-104 substrate (1 mg/ml in DEA buffer) was added to each well and reacted at room temperature. The plate was read on a Multiskan MCC/340 at 405 nM: with the 620nM absorbance subtracted.

Results

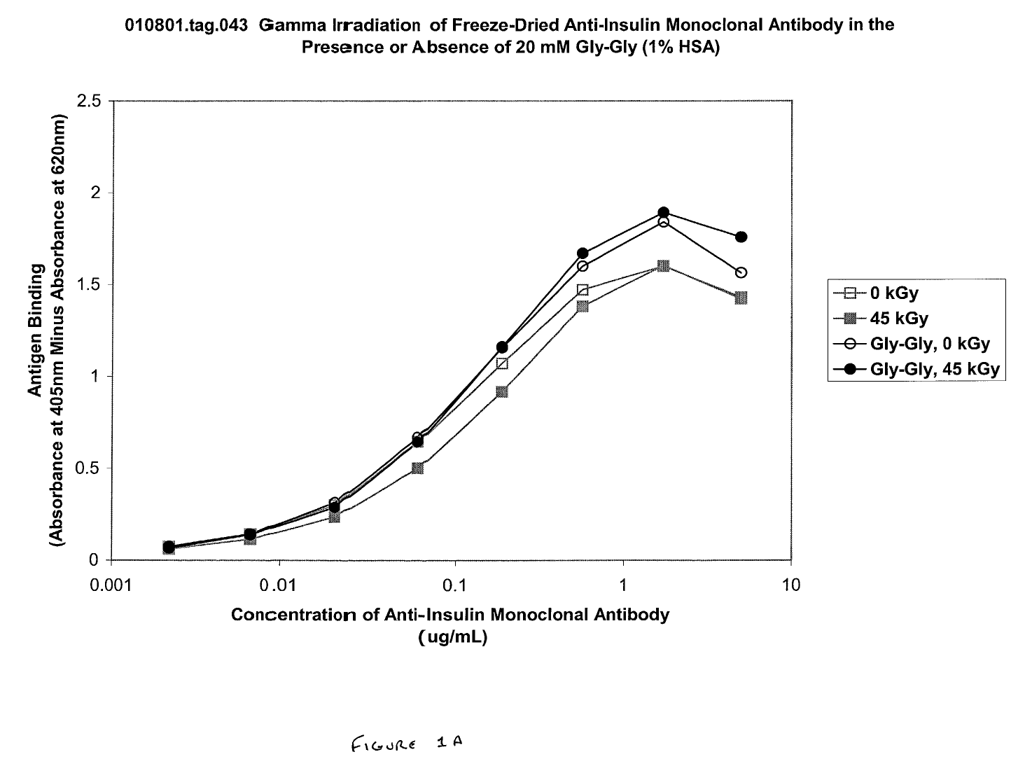

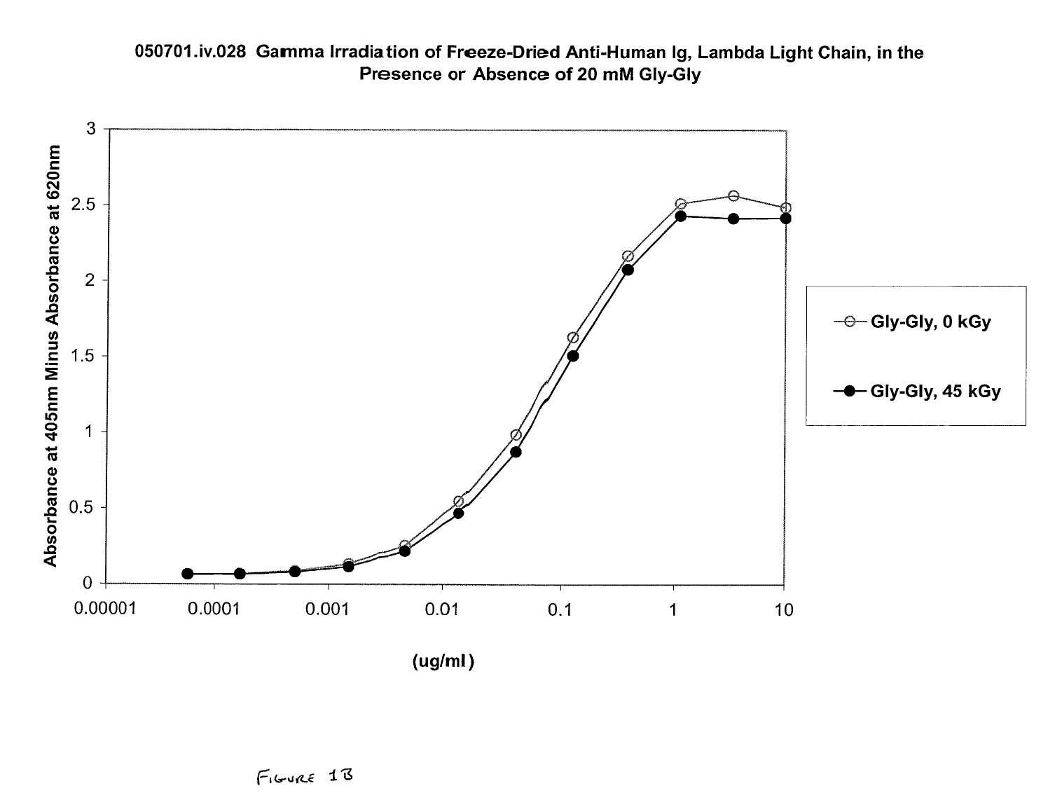

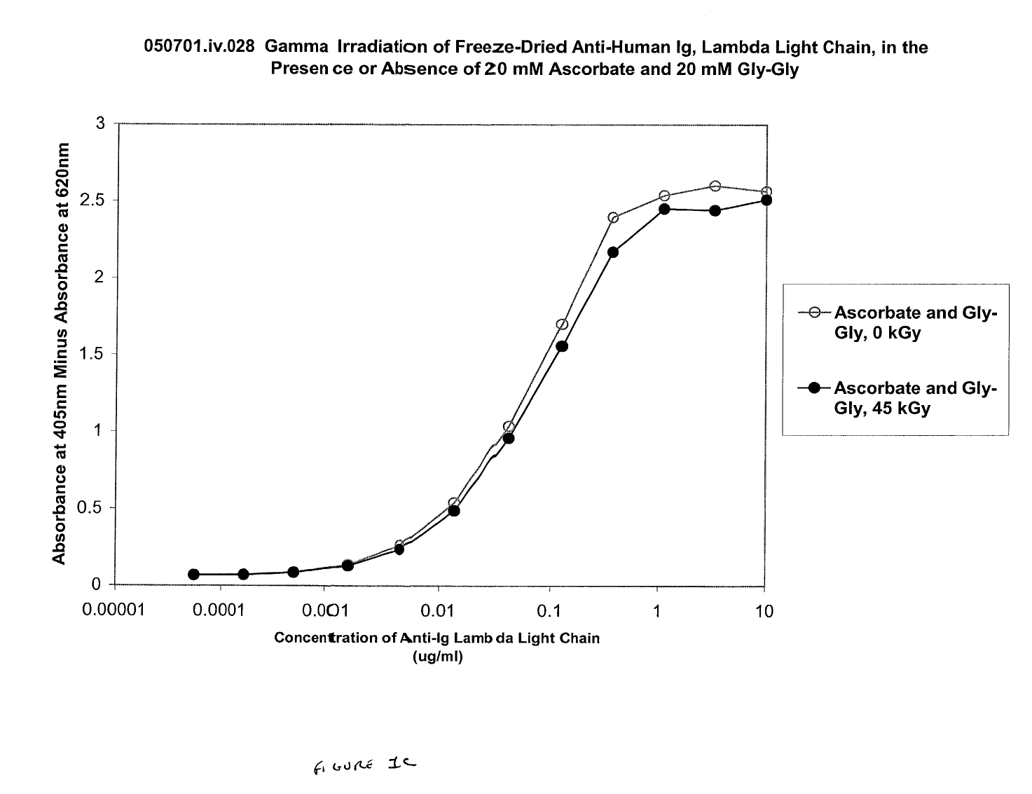

As shown in FIG. 1A, freeze-dried anti-insulin monoclonal immunoglobulin, supplemented with 1% HSA, gamma irradiated to 45 kGy resulted in an average loss in activity of 1.5 fold (average loss in avidity of 33%). Samples irradiated to 45 kGy in the presence of the dipeptide Gly—Gly (20 mM) showed 100% recovery of activity. Unirradiated samples containing the dipeptide Gly—Gly (20 mM) also showed ˜100% recovery of activity.

Adding 5% sucrose to freeze-dried anti-insulin monoclonal immunoglobulin containing 1% HSA resulted in an average recovery of 70% of the activity in the sample irradiated to 45 kGy (average loss in activity of approximately 1.5 fold or approximately 30% loss in avidity). Samples irradiated to 45 kGy in the presence of Gly—Gly showed ˜79% recovery of activity.

As shown in FIGS. 1B-1C, similar results have been obtained upon the addition of 20 mM Gly—Gly or the combination of ascorbate (20 mM) and Gly—Gly (20 mM) to another monoclonal IgG biological material of different specificity (anti-Ig Lambda Light Chain).

### Example 2

In this experiment, the protective effect of Gly—Gly (20 mM) on lyophilized anti-insulin monoclonal immunoglobulin was evaluated.

Method

In 3 ml glass vials, 1.0 ml total volume containing 100 μg anti-insulin monoclonal immunoglobulin, with 10 mg BSA (1%) and either no stabilizer or the stabilizer of interest was lyophilized. Samples were irradiated with gamma radiation (45 kGy total dose, dose rate 1.83 kGy/hr, temperature 4° C.) and then reconstituted with 1 ml of water. Karl Fischer moisture analysis was performed on the quadruplicate samples that did not contain immunoglobulin.

Immunoglobulin binding activity of independent duplicate samples was determined by a standard ELISA protocol: Maxisorp plates were coated overnight with 2.5 μg/ml insulin antigen. Three-fold serial dilutions of anti-insulin monoclonal immunoglobulin samples starting at 5 μg/ml were used. Goat anti-mouse phosphatase conjugate was used at 50 mg/ml. Relative potency values of irradiated samples compared to their corresponding unirradiated sample were calculated using the parallel line analysis software package (PLA 1.2 from Stegmann Systemberatung). Mass spectroscopy analysis was performed by M-scan, Inc. of Westchester Pa.

Results

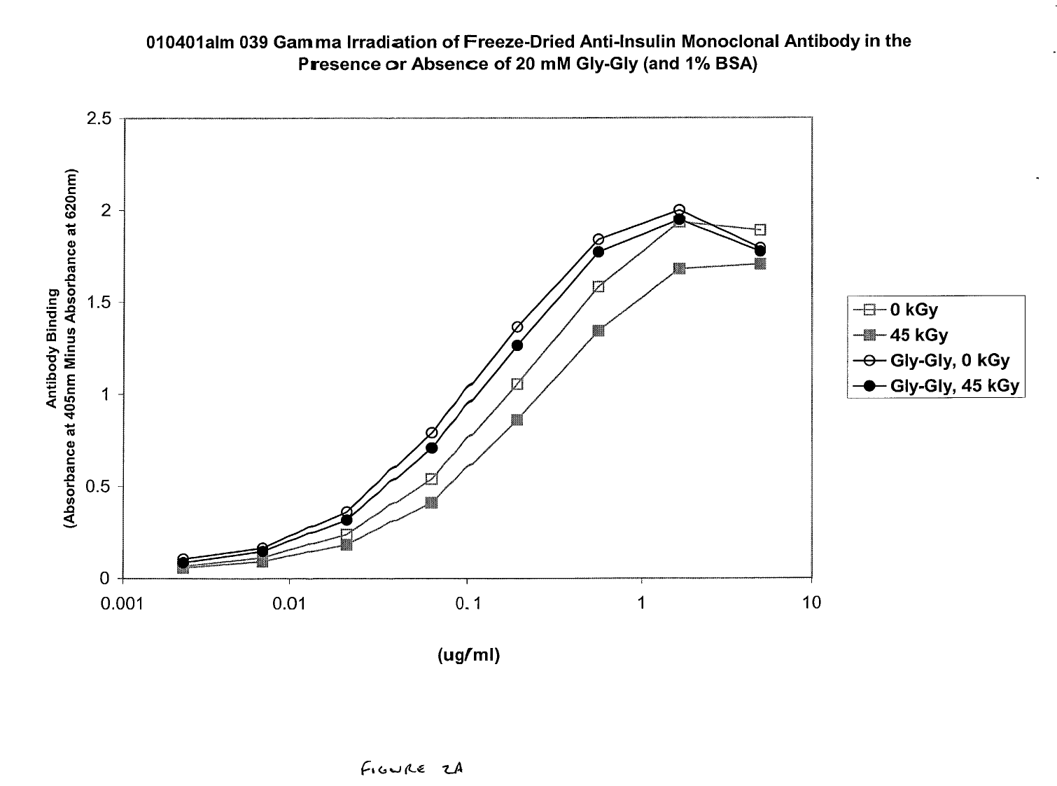

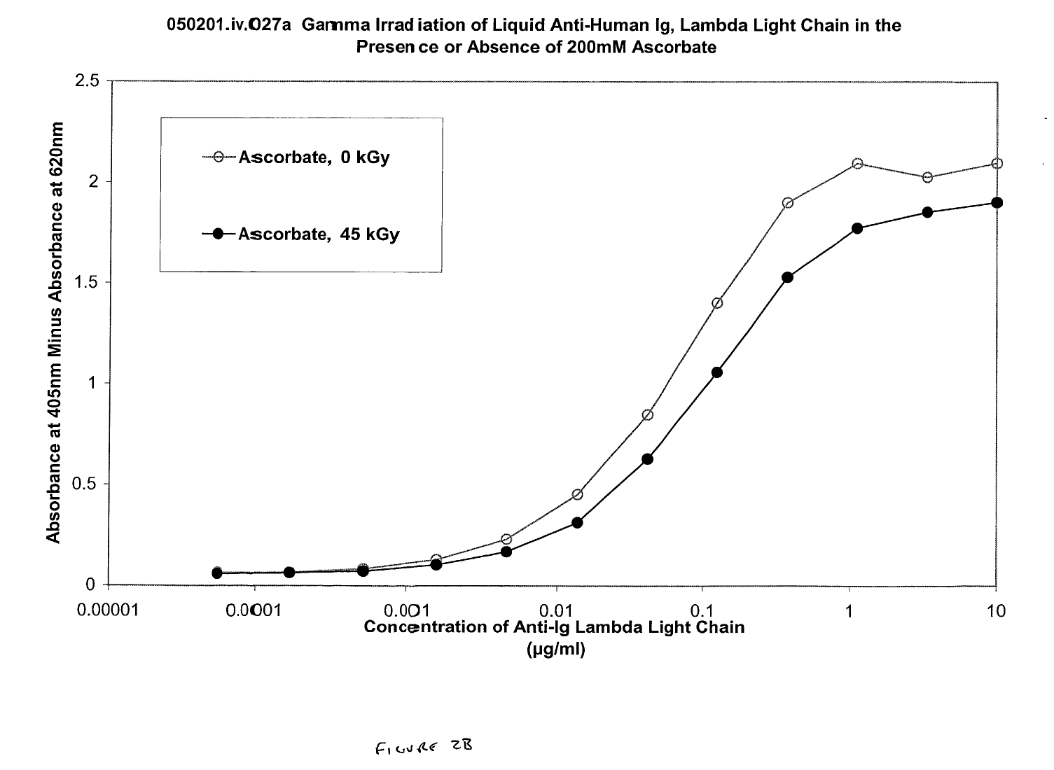

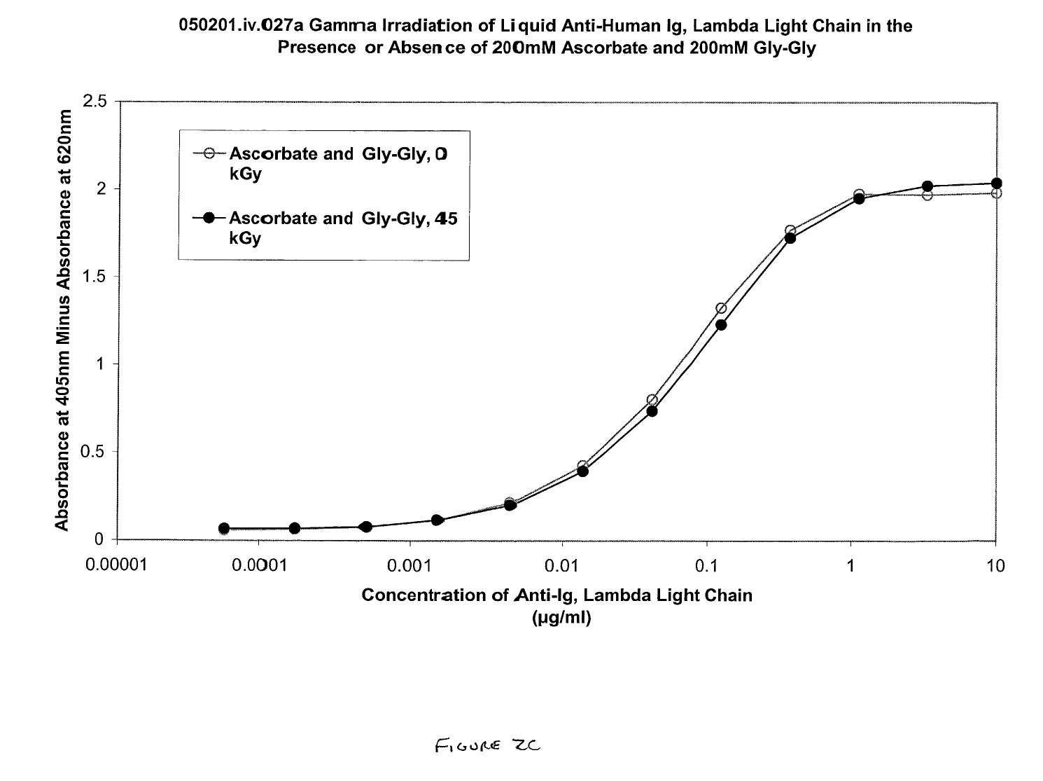

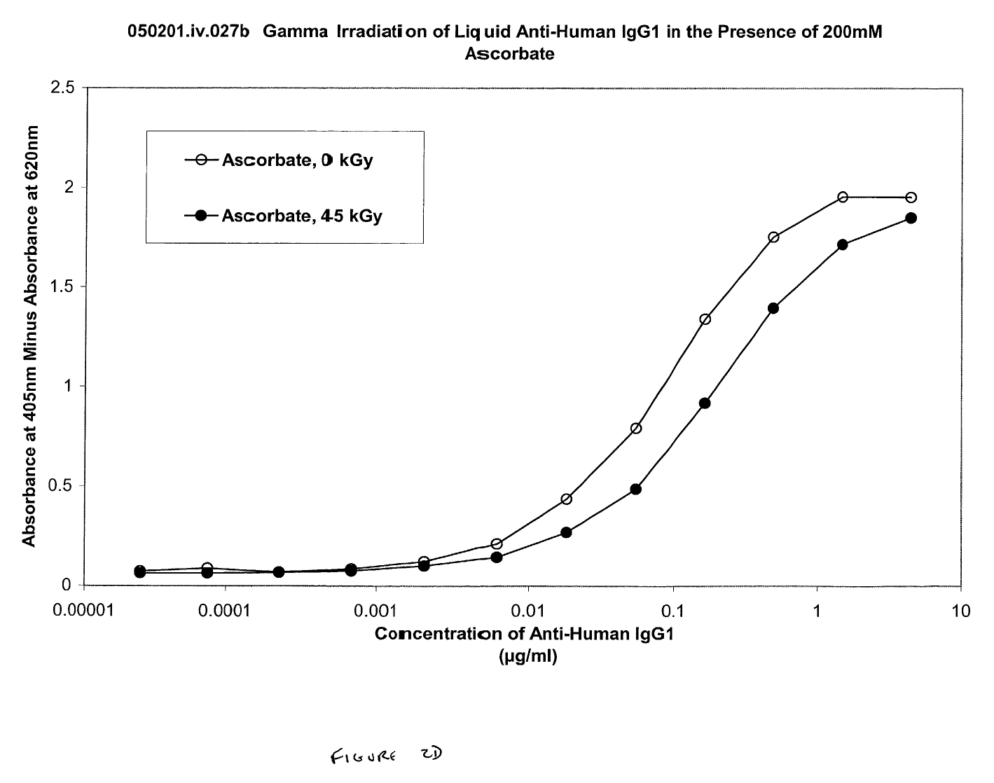

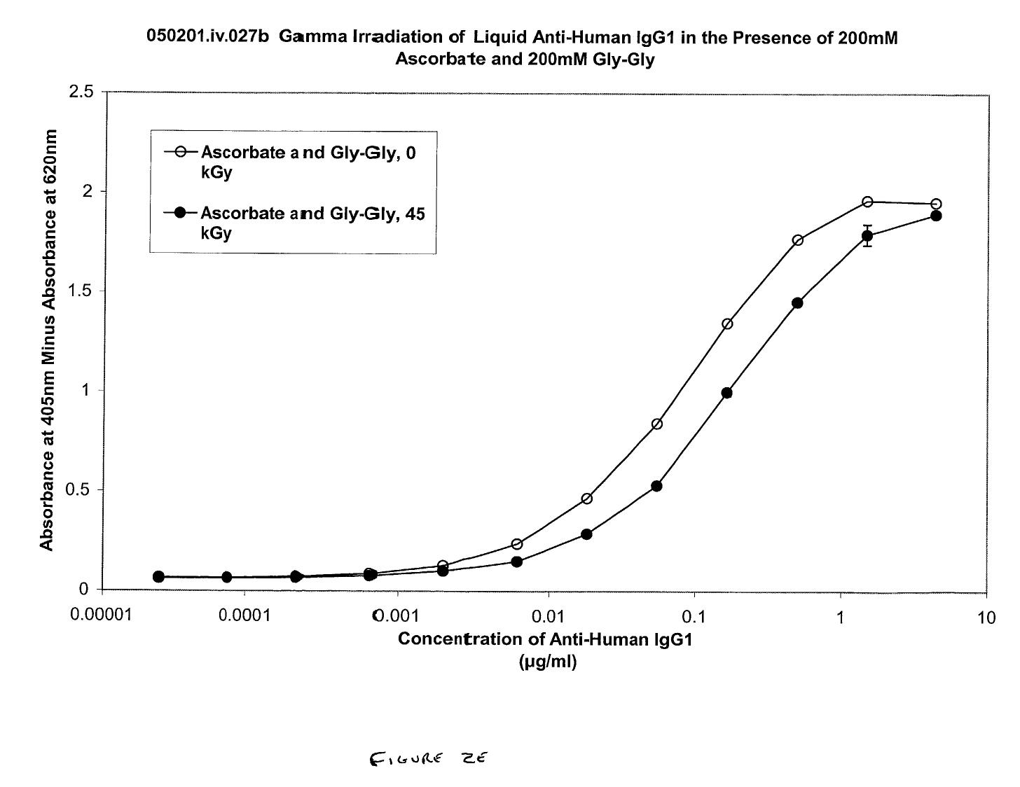

As illustrated in FIG. 2A, irradiation of lyophilized anti-insulin monoclonal immunoglobulin in the presence of 1% bovine serum albumin resulted in the loss of approximately 30% avidity (relative to unirradiated samples) of the immunoglobulin for its immobilized antigen. The addition of the dipeptide Gly—Gly resulted in recovery of 77-84% avidity.

As shown in FIGS. 2B-2E, similar results have been obtained upon the addition of 200 mM ascorbate or the combination of ascorbate (200 mM) and Gly—Gly (200 mM) to two other monoclonal IgG preparations of different specificity (anti-Ig Lambda Light Chain and anti-IgGI).

### Example 3

In this experiment, the protective effect of ascorbate (200 mM), alone or in combination with Gly—Gly (200 mM), on a liquid polyclonal antibody preparation was evaluated.

Method

In 2 ml glass vials, samples of IGIV (50 mg/ml) were prepared with either no stabilizer or the stabilizer of interest. Samples were irradiated with gamma radiation (45 kGy total dose, dose rate 1.8 kGy/hr, temperature 4° C.) and then assayed for functional activity and structural integrity.

Functional activity of independent duplicate samples was determined by measuring binding activity for rubella, mumps and CMV using the appropriate commercial enzyme immunoassay (EIA) kit obtained from Sigma, viz., the Rubella IgG EIA kit, the Mumps IgG EIA kit and the CMV IgG EIA kit.

Structural integrity was determined by gel filtration (elution buffer: 50 mM NaPi, 100 mM NaCl, pH 6.7; flow rate: 1 ml/min; injection volume 50 μl) and SDS-PAGE (pre-cast tris-glycine 4-20% gradient gel from Novex in a Hoefer Mighty Small Gel Electrophoresis Unit running at 125V; sample size: 10 μl).

Results

Functional activity

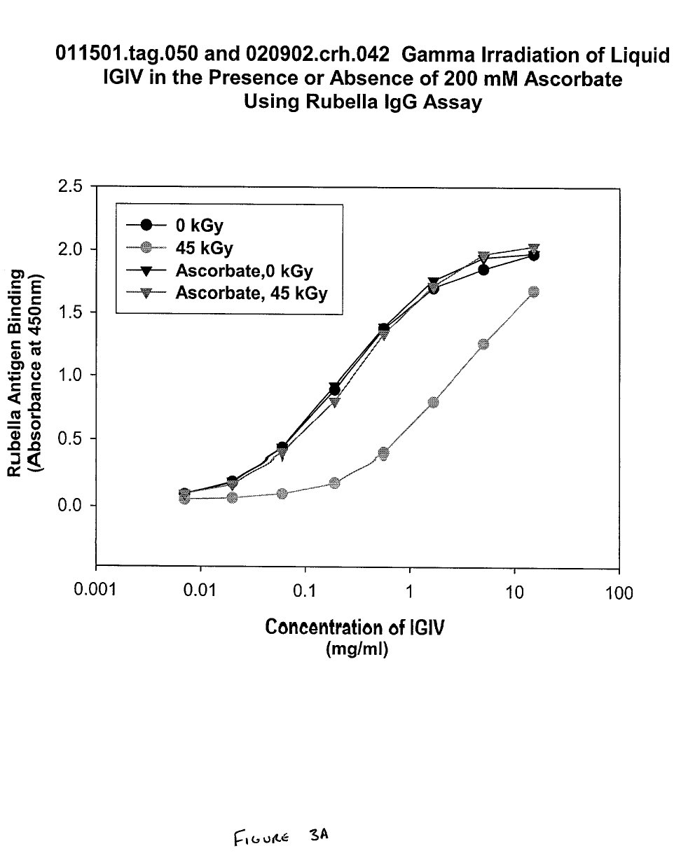

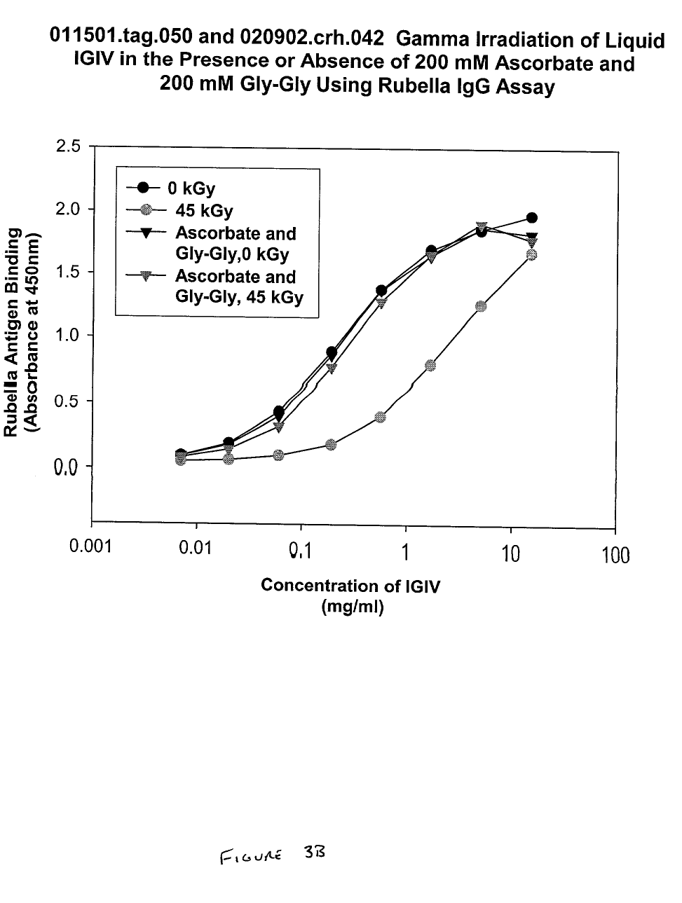

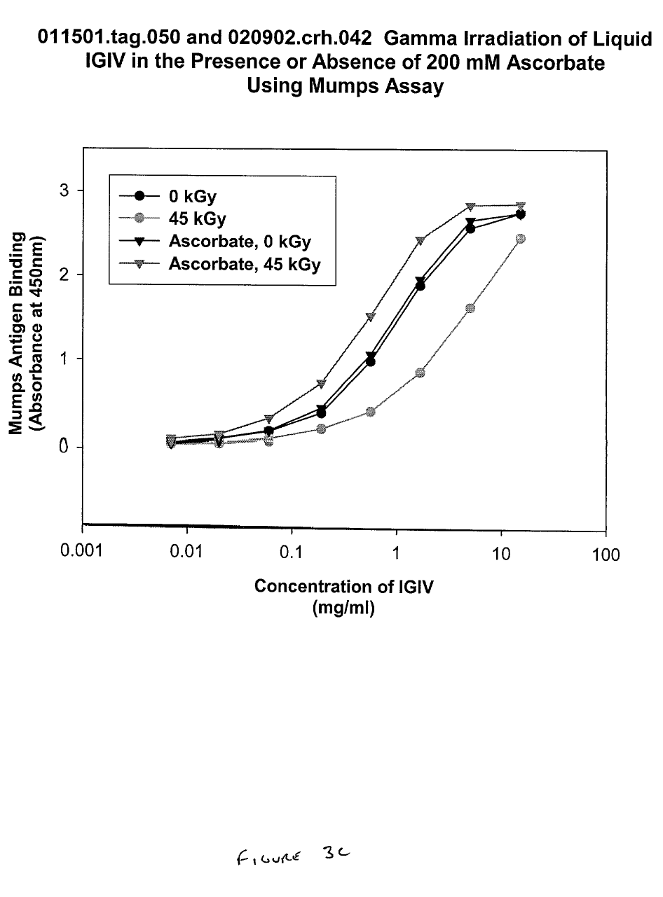

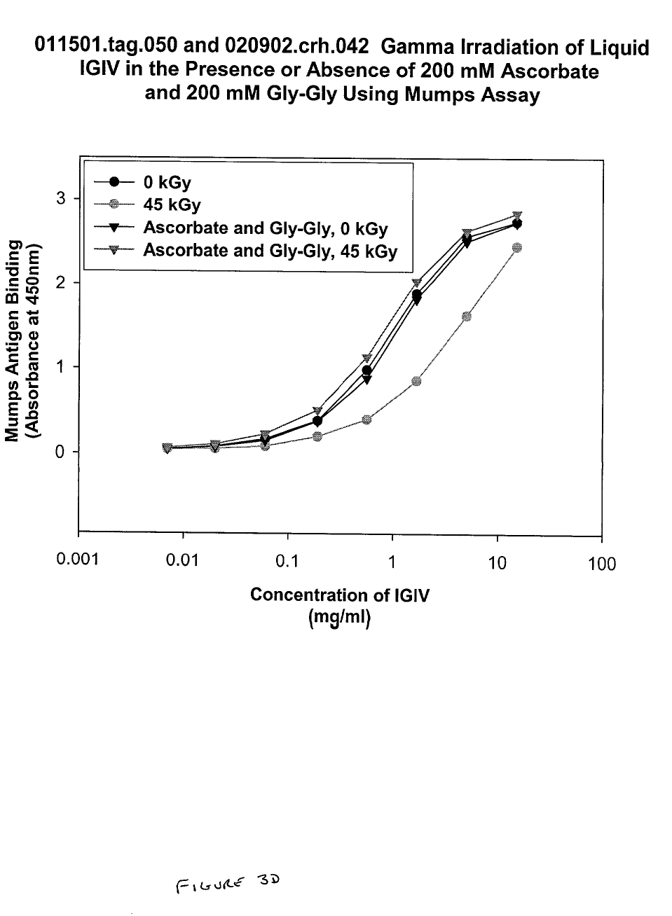

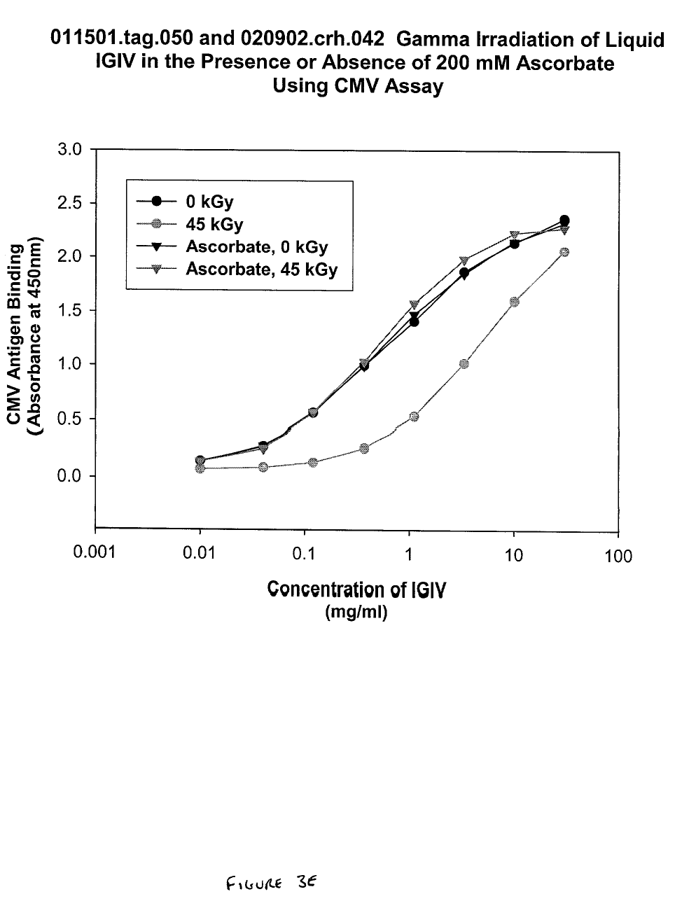

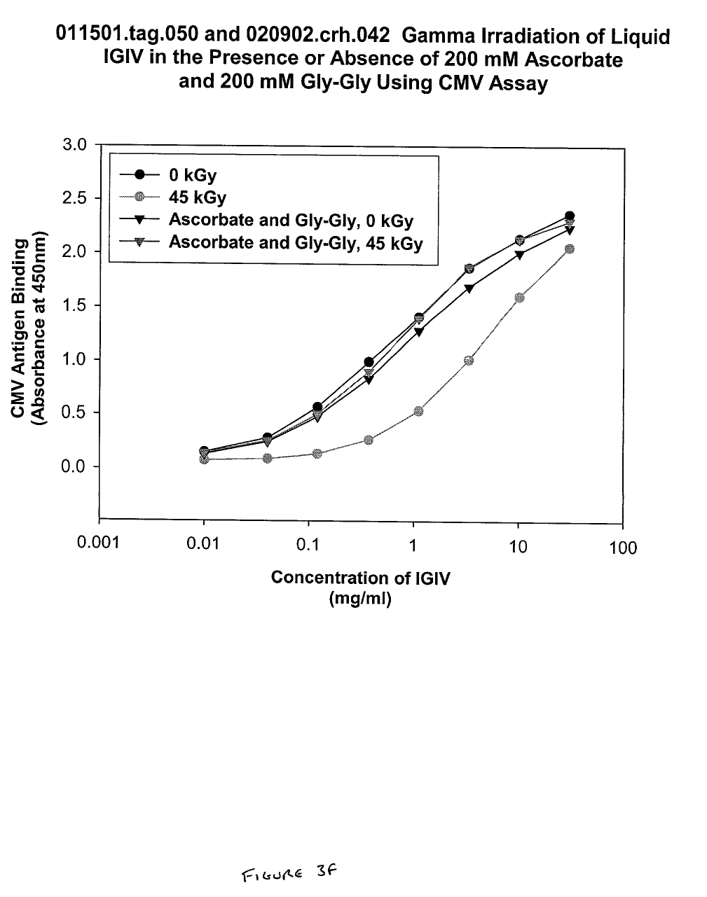

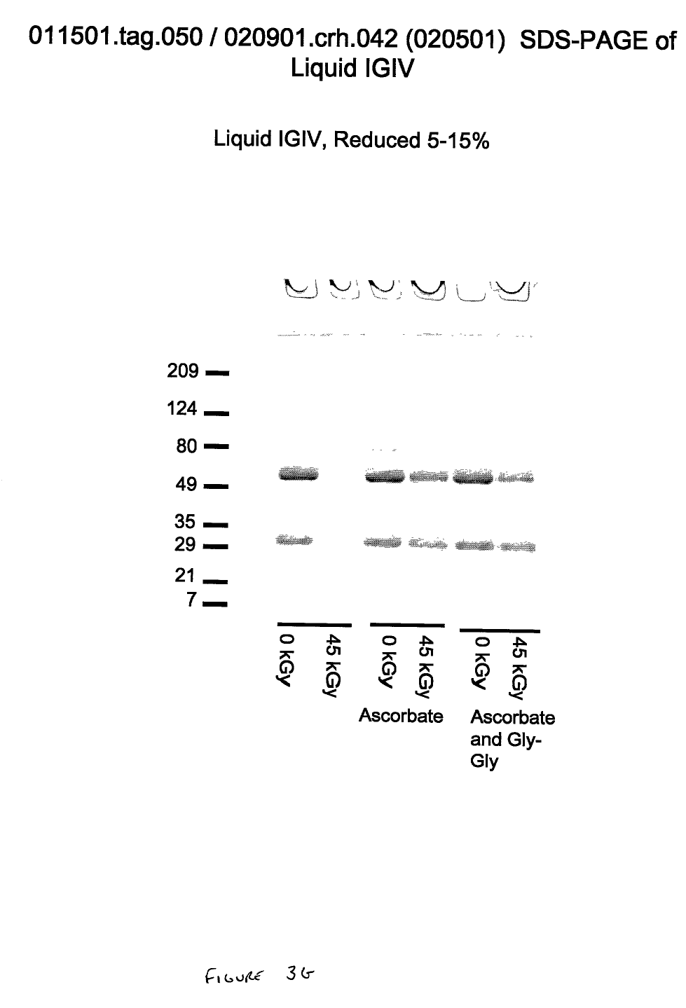

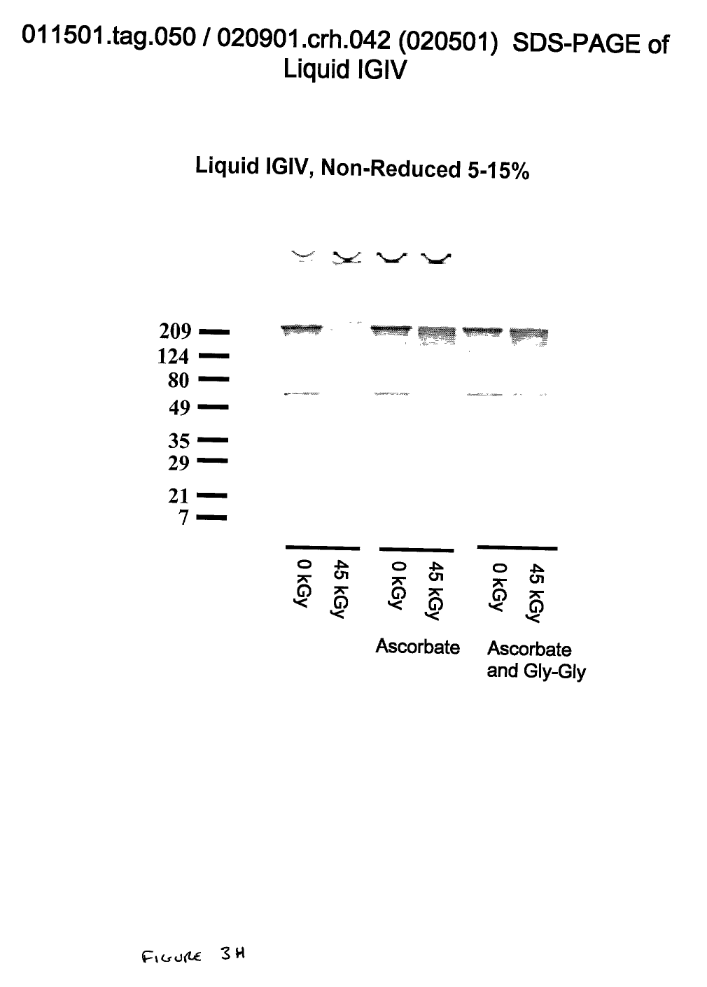

As illustrated in FIGS. 3A-3B, irradiation of liquid polyclonal antibody samples to 45 kGy resulted in the loss of approximately 1 log of activity for rubella (relative to unirradiated samples). The addition of ascorbate alone improved recovery, as did the addition of ascorbate in combination with the dipeptide Gly—Gly.

Similarly, as illustrated in FIGS. 3C-3D, irradiation of liquid polyclonal antibody samples to 45 kGy resulted in the loss of approximately 0.5-0.75 log of activity for mumps. The addition of ascorbate alone improved recovery, as did the addition of ascorbate in combination with the dipeptide Gly—Gly.

Likewise, as illustrated in FIGS. 3E-3F, irradiation of liquid polyclonal antibody samples to 45 kGy resulted in the loss of approximately 1 log of activity for CMV. The addition of ascorbate alone improved recovery, as did the addition of ascorbate in combination with the dipeptide Gly—Gly.

Structural analysis

Liquid polyclonal antibody samples irradiated to 45 kGy in the absence of a stabilizer showed significant loss of material and evidence of both aggregation and fragmentation. The irradiated samples containing ascorbate or a combination of ascorbate and the dipeptide Gly—Gly exhibited only slight breakdown and some aggregation as demonstrated by gel filtration and SDS-PAGE (FIGS. 3G-3H).

### Example 4

In this experiment, the protective effect of ascorbate (20 mM) and/or Gly—Gly (20 mM) on lyophilized anti-insulin monoclonal immunoglobulin irradiated at a high dose rate was evaluated.

Method

Samples were freeze-dried for approximately 64 hours and stoppered under vacuum and sealed with an aluminum, crimped seal. Samples were irradiated at a dose rate of 30 kGy/hr to a total dose of 45 kGy at 4° C.

Monoclonal immunoglobulin activity was deterrnined by a standard ELISA protocol. Maxisorp plates were coated with human recombinant insulin at 2.5 μg/ml overnight at 4° C. The plate was blocked with 200 III of blocking buffer (PBS, pH 7.4, 2% BSA) for two hours at 37° C. and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed six times with wash buffers. One hundred μl of Sigma-104 substrate (1 mg/ml in DEA buffer) was added to each well and reacted at room temperature. The plate was read on a Multiskan MCC/340 at 405 nM with the 620 nM absorbance subtracted.

Results

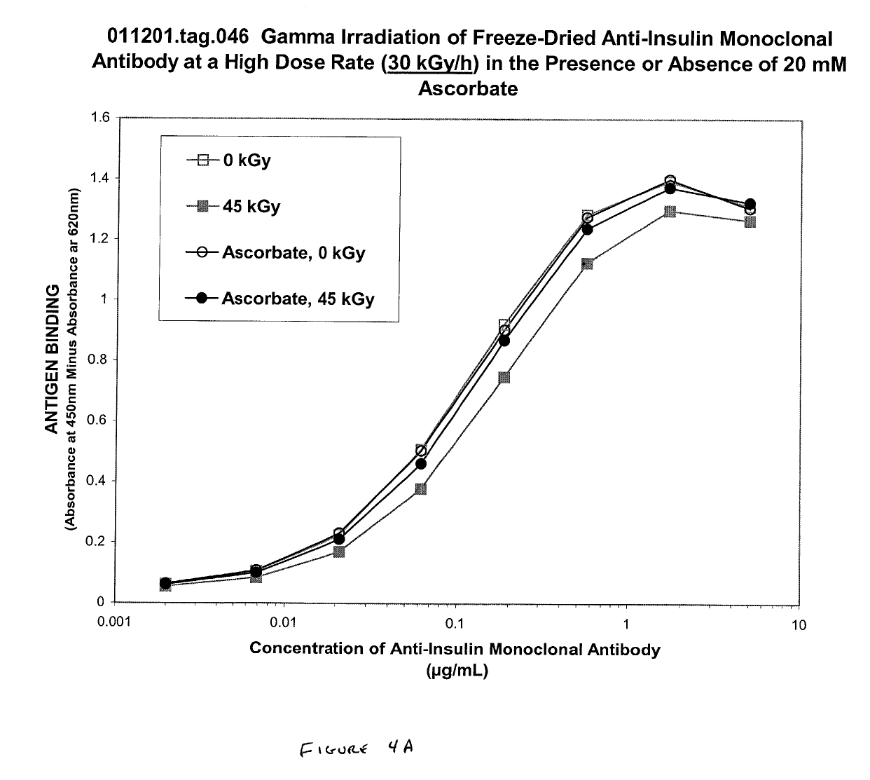

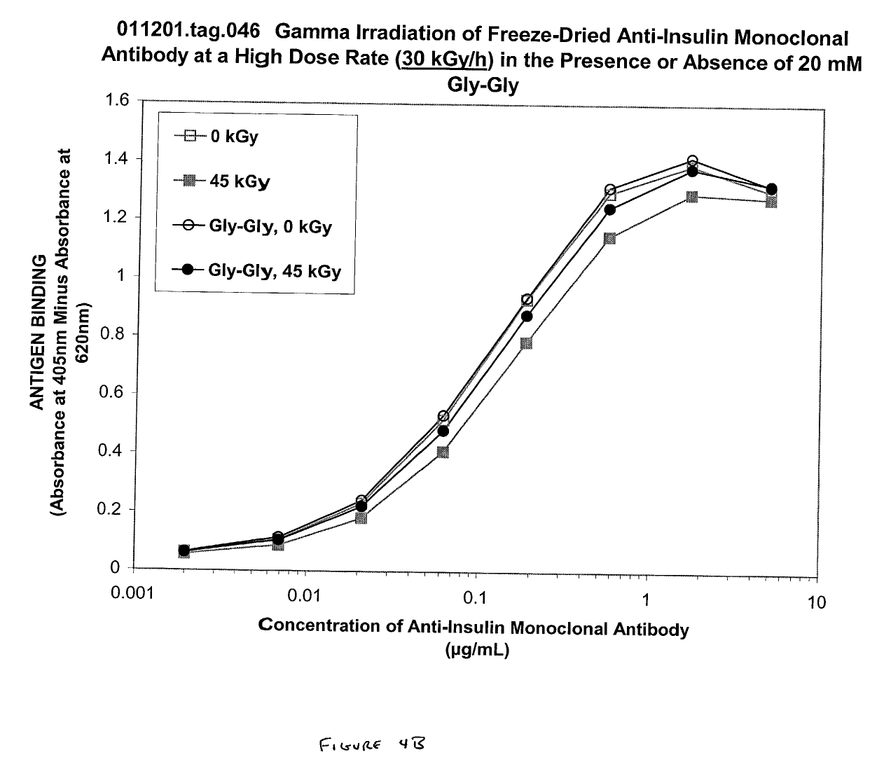

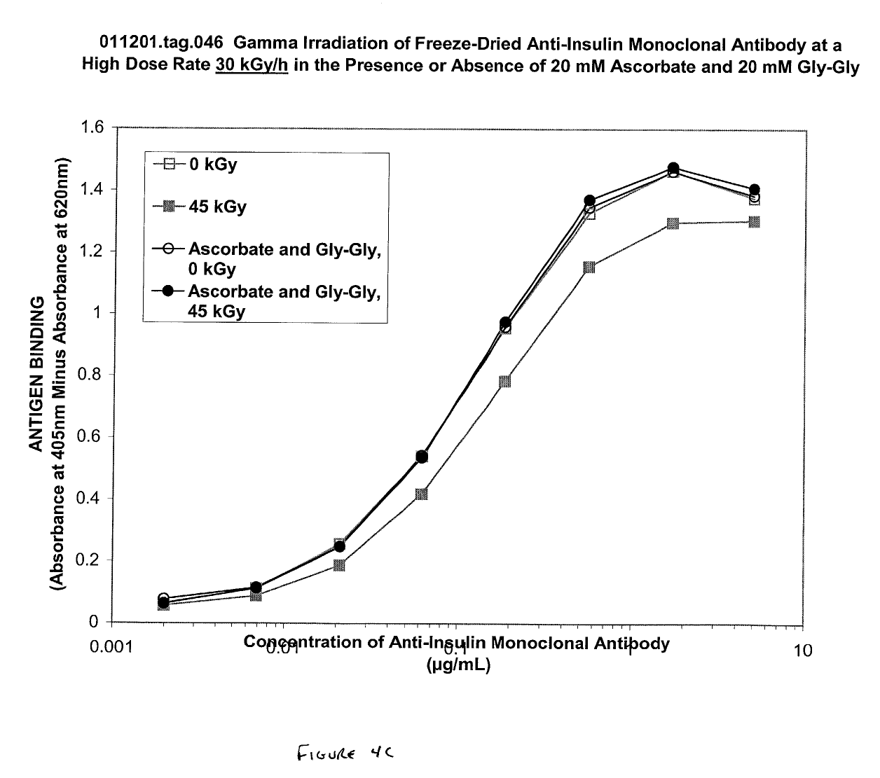

As shown in FIGS. 4A-4C, freeze-dried anti-insulin monoclonal immunoglobulin gamma irradiated to 45 kGy resulted in an average loss in activity of ˜32% (average loss in avidity of ˜1.5 fold).

Lyophilized anti-insulin monoclonal imnuunoglobulin samples irradiated to 45 kGy in the presence of 20 mM ascorbate had only a 15% loss in activity (˜1.1 fold loss in avidity), and those samples irradiated to 45 kGy in the presence of 20 mM Gly—Gly had only a 23% loss in activity (˜1.3 fold loss in avidity). Lyophilized anti-insulin monoclonal immunoglobulin samples irradiated to 45 kGy in the presence of 20 mM ascorbate and 20 mM Gly—Gly showed no loss in activity (no loss in avidity).

### Example 5

In this experiment, the protective effect of ascorbate (200 mM) and/or Gly—Gly (200 mM) on liquid anti-insulin monoclonal immunoglobulin irradiated to 45 kGy.

Method

Liquid samples containing 100 μg antibody (2 mg/ml) with 10% BSA were irradiated at a dose rate of 1.83-1.88 kGy/hr to a total dose of 45.1-46.2 kGy at 4° C.

Monoclonal immunoglobulin activity was determined by a standard ELISA protocol. Maxisorp plates were coated with human recombinant insulin at 2.5 μg/ml overnight at 4° C. The plate was blocked with 200 μl of blocking buffer (PBS, pH 7.4, 2% BSA) for two hours at 37° C. and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed six times with wash buffers. One hundred μl of Sigma-104 substrate (1 mg/ml in DEA buffer) was added to each well and reacted at room temperature. The plate was read on a Multiskan MCC/340 at 405 nM with the 620 nM absorbance subtracted.

Results

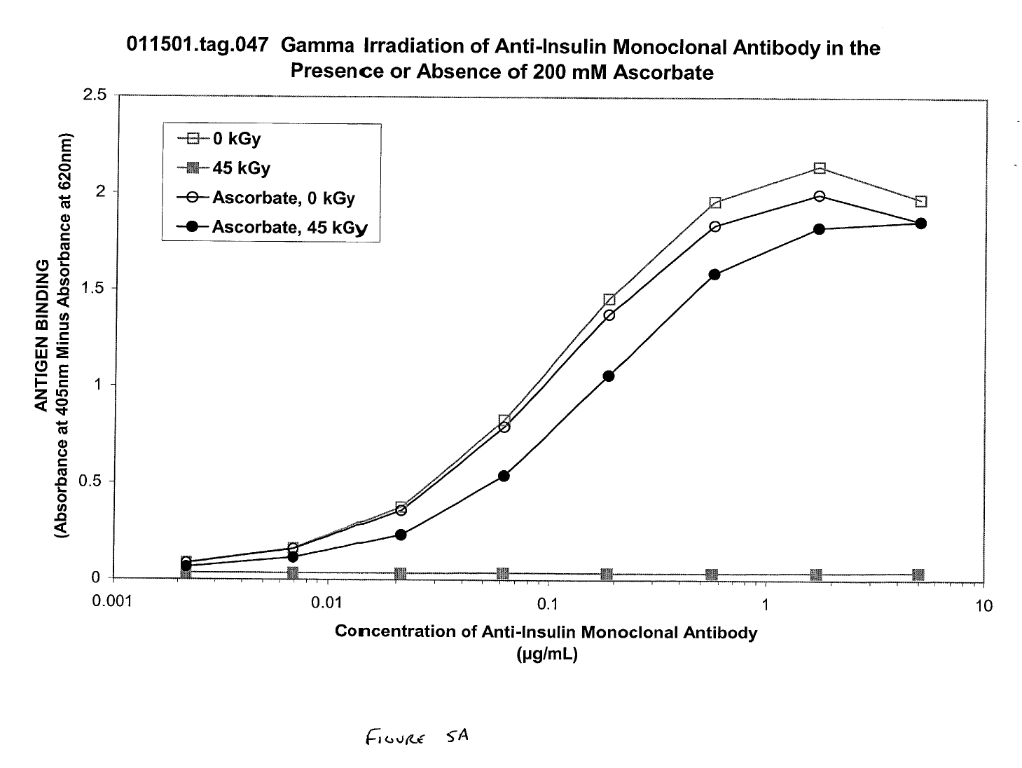

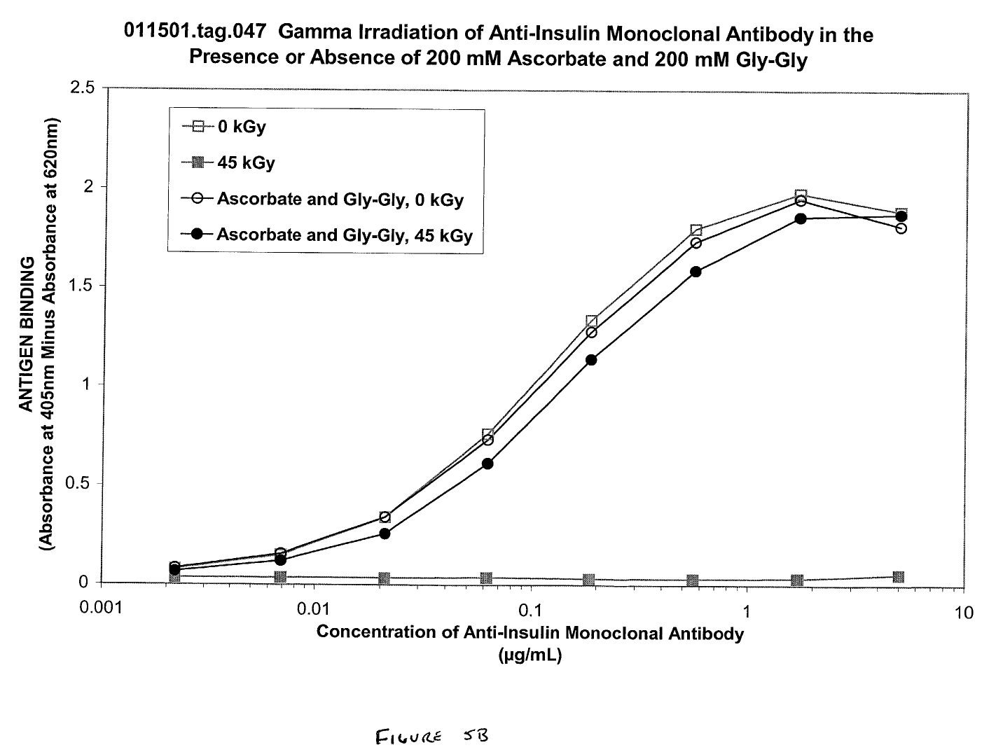

As shown in FIGS. 5A-5B, liquid anti-insulin mnonoclonal immunoglobulin gamma irradiated to 45 kGy resulted in a complete loss of activity.

Liquid anti-insulin monoclonal immunoglobulin samples irradiated to 45 kGy in the presence of 200 mM ascorbate had a 48% loss in activity compared to control. Liquid anti-insulin monoclonal immuno globulin samples irradiated to 45 kGy in the presence of both 200 mM ascorbate and 200 mM Gly—Gly showed only a 29% loss in activity.

### Example 6

In this experiment, the protective effect of ascorbate (200 mM) and a combination of ascorbate (200 mM) and Gly—Gly (200 mM) on two different frozen enzyme preparations (a glycosidase and a sulfatase) was evaluated.

Method

In glass vials, 300 μl total volume containing 300 μg of enzyme (1 mg/ml) were prepared with either no stabilizer or the stabilizer of interest. Samples were irradiated with gamma radiation (45 kGy total dose, dose rate and temperature of either 1.616 kGy/hr and 21.5° C. or 5.35 kGy/hr and −21.9° C.) and then assayed for structural integrity.

Structural integrity was determined by SDS-PAGE. Three 12.5% gels were prepared according to the following recipe: 4.2 ml acrylamide; 2.5 ml 4X-Tris (pH 8.8); 3.3 ml water; 100 μl 10% APS solution; and 10 μl TEMED, and placed in an electrophoresis unit with 1X Running Buffer (15.1 g Tris base; 72.0 g glycine; 5.0 g SDS in 1 l water, diluted 5-fold). Irradiated and control samples (1 mg/ml) were diluted with Sample Buffer (+/− beta-ME) in Eppindorf tubes and then centrifuged for several minutes. 20 μl of each diluted sample (˜10 μg) were assayed.

Results

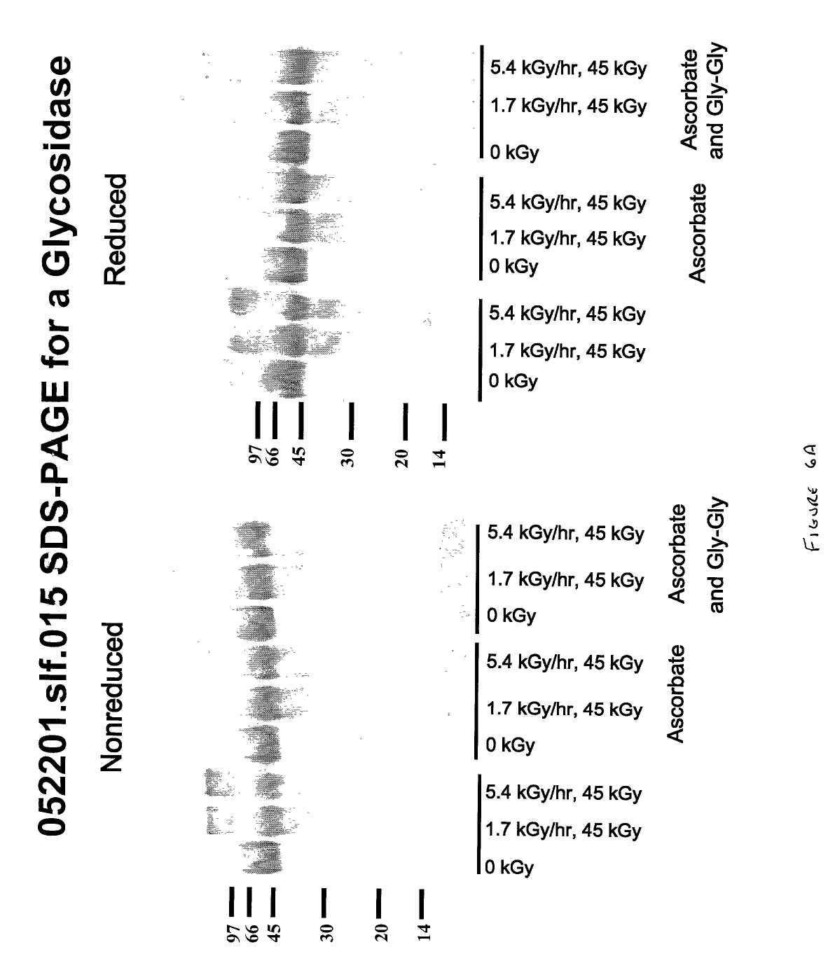

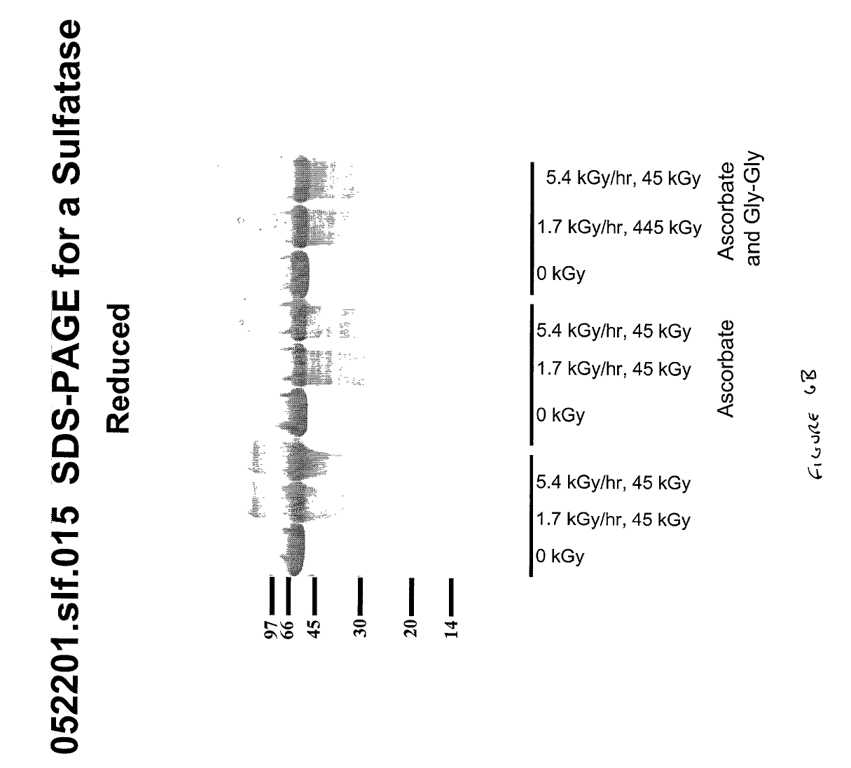

As shown in FIG. 6A, liquid glycosidase samples irradiated to 45 kGy in the absence of a stabilizer showed significant loss of material and evidence of both aggregation and fragmentation. Much greater recovery of material was obtained from the irradiated samples containing ascorbate or a combination of ascorbate and Gly—Gly.

As shown in FIG. 6B, liquid sulfatase samples irradiated to 45 kGy in the absence of a stabilizer showed significant loss of material and evidence of both aggregation and fragmentation. Much greater recovery of material was obtained from the irradiated samples containing ascorbate or a combination of ascorbate and Gly—Gly.

### Example 7

In this experiment, the protective effect of ascorbate (200 mM) and a combination of ascorbate (200 mM) and Gly—Gly (200 mM) on a frozen glycosidase preparation was evaluated.

Method

Samples were prepared in 2 ml glass vials containing 52.6 μl of a glycosidase solution (5.7 mg/ml), no stabilizer or the stabilizer(s) of interest and sufficient water to make a total sample volume of 300 μl. Samples were irradiated at a dose rate of 1.616 or 5.35 kGy/hr at a temperature between −20 and −21.9° C. to a total dose of 45 kGy.

Structural integrity was determined by reverse phase chromatography. 10 μl of sample were diluted with 90 μl solvent A and then injected onto an Aquapore RP-300 (c-8) column (2.1×30 mm) mounted in an Applied Biosystems 130A Separation System Microbore HPLC. Solvent A: 0.1% trifluoroacetic acid; solvent B: 70% acetonitrile, 30% water, 0.085% trifluoroacetic acid.

Results

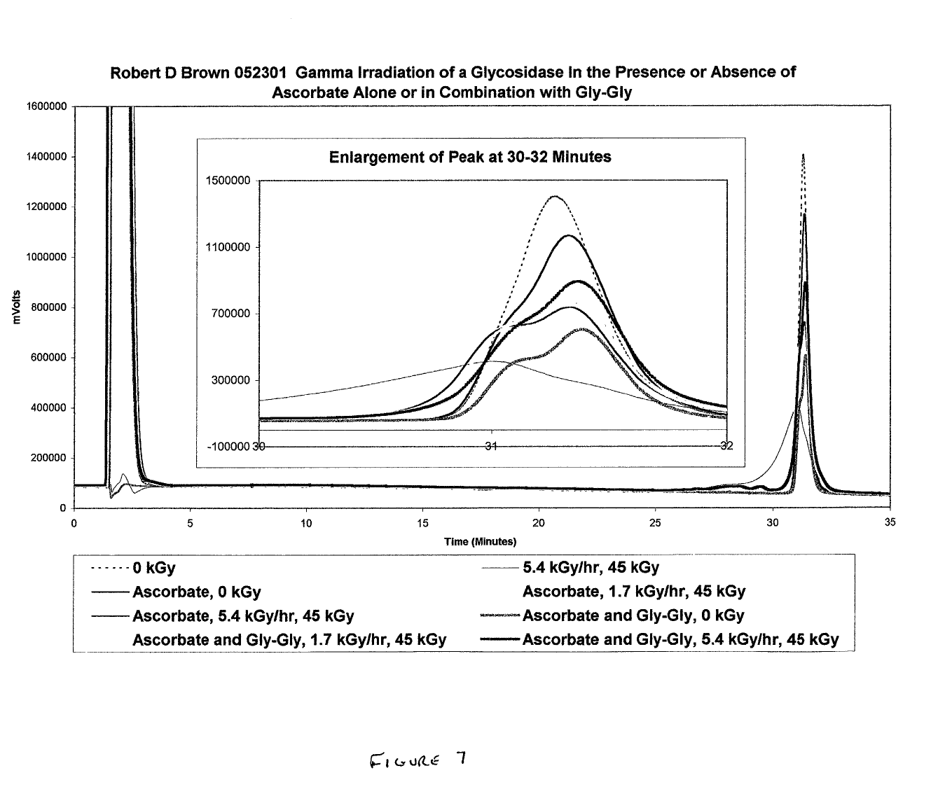

Liquid enzyme samples irradiated to 45 kGy in the absence of a stabilizer showed broadened and reduced peaks. As shown in FIG. 7, much greater recovery of material, as evidenced by significantly less reduction in peak size compared to control, was obtained from the irradiated samples containing ascorbate or a combination of ascorbate and Gly—Gly.

### Example 8

In this experiment, the protective effect of various stabilizers on anti-insulin monoclonal immunoglobulin (50 mg/ml) supplemented with 0.1% human serum albumin (HSA) exposed to gamma irradiation up to 100 kGy was evaluated. The stabilizers tested were ascorbate (200 mM) and a mixture of ascorbate (200 mM) and Gly—Gly (200 mM).

Methods

Samples were irradiated at a dose rate of 0.458 kGy/hr to a total dose of 25, 50 or 100 kGy at ambient temperature (20-25° C.).

Monoclonal immunoglobulin activity was determined by a standard ELISA protocol. Maxisorp plates were coated with human recombinant insulin at 2.5 μg/ml overnight at 4° C. The plate was blocked with 380 μl of blocking buffer (PBS, pH 7.4, 2% BSA) for two hours at 37° C. and then washed three times with wash buffer (TBS, pH 7, 0.05% TWEEN 20). Serial 3-fold dilutions were performed. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed eight times with wash buffers. One hundred lii of Sigma-104 substrate (1 mg/ml in DEA buffer) was added to each well and reacted at room temperature. The plate was read on a Multiskan MCC/340 at 405 nM with the 620 nM absorbance subtracted.

Results

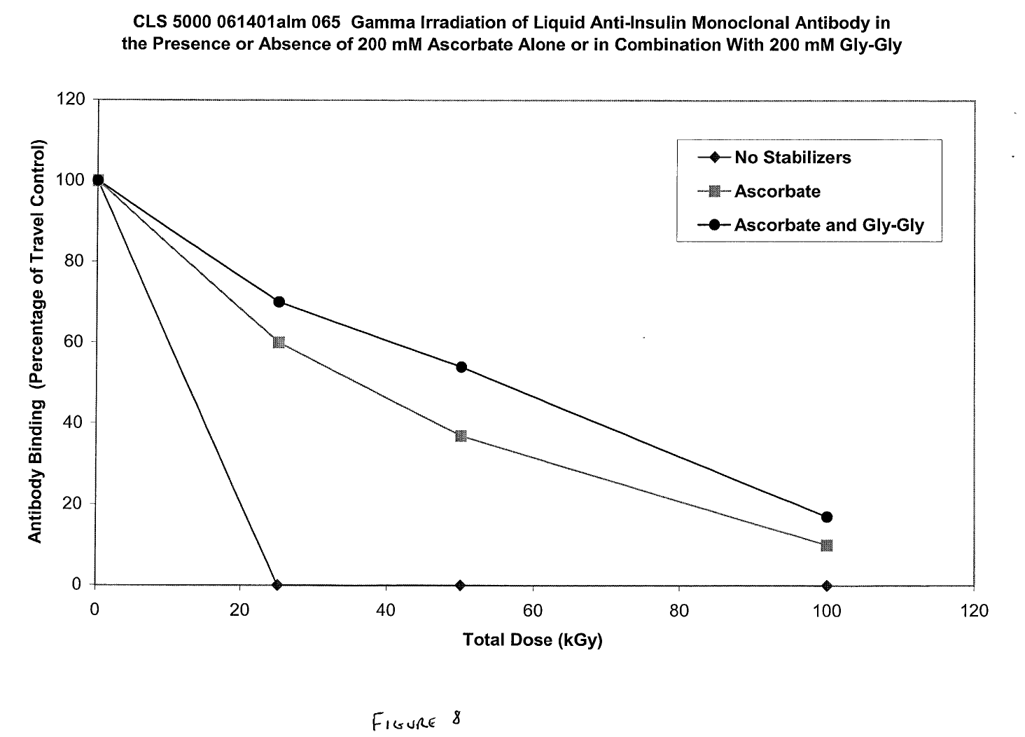

As shown in FIG. 8, samples of anti-insulin monoclonal immunoglobulin supplemented with 1% HSA lost all binding activity when gamma irradiated to 25 kGy. In contrast, samples containing a combination of ascorbate and Gly—Gly retained about 67% of binding activity when irradiated to 25 kGy, 50% when irradiated to 50 kGy and about 33% when irradiated to 100 kGy. Samples containing ascorbate alone retained about 65% of binding activity when irradiated to 25 kGy, about 33% when irradiated to 50 kGy and about 12% when irradiated to 100 kGy.

### Example 9

In this experiment, the protective effect of the dipeptide stabilizer L-camosine, alone or in combination with ascorbate (50 mM), on gamma irradiated liquid urokinase was evaluated.

Methods

Liquid urokinase samples (2000 IU/ml) were prepared using a buffer solution containing 100 mM Tris pH 8.8, 100 mM NaCl, and 0.2% PEG 8000. Samples were irradiated at a dose rate of 1.92 kGy/hr to a total dose of 45 kGy at 4° C.

Urokinase activity was determined using a colorimetric assay. The substrate was Urokinase Substrate I, Colorimetric, Calbiochem 672157 lot B23901. Substrate was reconstituted in a buffer solution containing 50 mM Tris pH 8.8, 50 mM NaCl and 0.1% PEG 8000 to a concentration of 1 mM). Irradiated samples were centrifuged (1-1.5×1000 RPM, Sorvall RT6000B Refrigerated Centrifuge with Sorvall rotor H1000B) for approximately 3 minutes and then 50 μl of substrate solution were added. The samples with added substrate were incubated at 37° C. with shaking and absorbance at 406-620 mn determined at 20 minute intervals beginning 5 minutes after addition of substrate to the sample.

Results

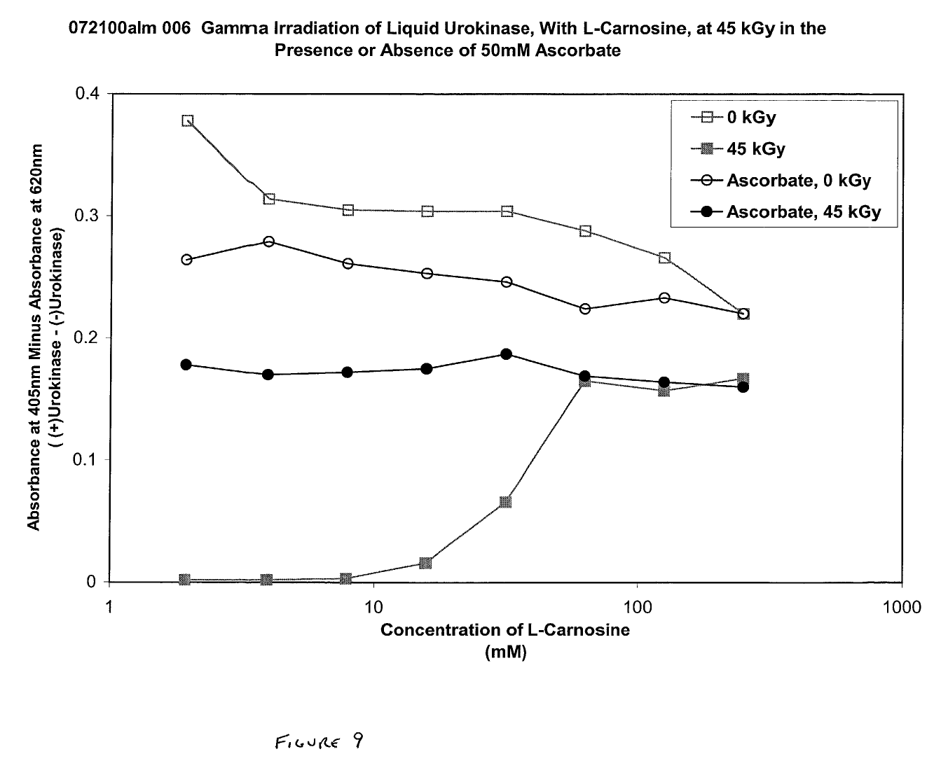

As shown in FIG. 9, L-carnosine showed a concentration dependent protection of liquid urokinase (from about 15 mM to about 62.5 mM) irradiated to a total dose of 45 kGy. At concentrations greater than 62.5 mM, no additional protective effect was observed. When L-carnosine was combined with ascorbate (50 mM), a protective effect on irradiated liquid urokinase was also observed.

### Example 10

In this experiment, the protective effect of the dipeptide stabilizer anserine on gamma irradiated liquid urokinase was evaluated.

Methods

Liquid urokinase samples (2000 IU/ml) were prepared using a buffer solution containing 100 mM Tris pH 8.8, 100 mM NaCl, and 0.2% PEG 8000. Samples were irradiated at a dose rate of 1.92 kGy/hr to a total dose of 45 kGy at 4° C.

Urokinase activity was determined using a colorimetric assay. The substrate was Urokinase Substrate I, Colorimetric, Calbiochem 672157 lot B23901. Substrate was reconstituted in a buffer solution containing 50 mM Tris pH 8.8, 50 mM NaCl and 0.1% PEG 8000 to a concentration of 1 mM). Irradiated samples were centrifuged (1-1.5×1000 RPM, Sorvall RT6000B Refrigerated Centrifuge with Sorvall rotor H1000B) for approximately 3 minutes and then 50 μl of substrate solution were added. The samples with added substrate were incubated at 37° C. with shaking and absorbance at 406-620 nm determined at 20 minute intervals beginning 5 minutes after addition of substrate to the sample.

Results

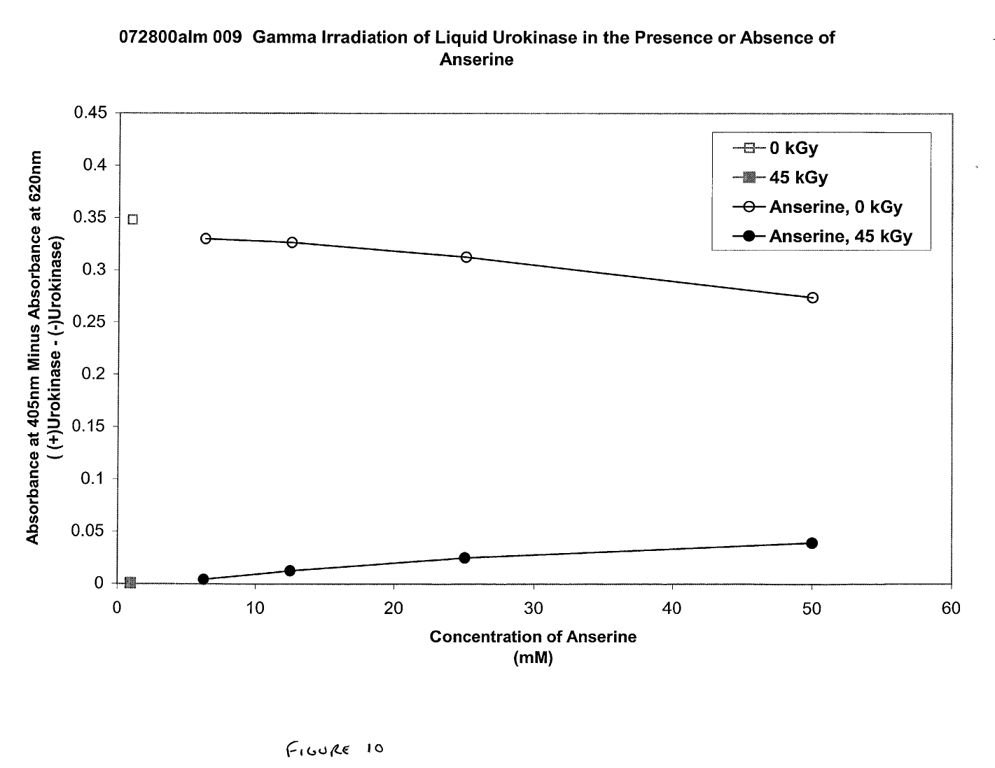

As shown in FIG. 10, the addition of anserine provided approximately 10-15% protection to liquid urokinase irradiated to a total dose of 45 kGy. In contrast, liquid urokinase samples containing no anserine showed a complete loss of activity.

### Example 11

In this experiment, the protective effect of L-carnosine on gamma irradiated liquid urokinase was evaluated.

Methods

Liquid urokinase samples (2000 IU/ml) were prepared using a buffer solution containing 100 mM Tris pH 8.8, 100 mM NaCl, and 0.2% PEG 8000. Samples were rradiated at a dose rate of 1.92 kGy/hr to a total dose of 45 kGy at 4° C.

Urokinase activity was determined using a colorimetric assay. The substrate was Urokinase Substrate I, Colorimetric, Calbiochem 672157 lot B23901. Substrate was reconstituted in a buffer solution containing 50 mM Tris pH 8.8, 50 mM NaCl and 0.1% PEG 8000 to a concentration of 1 mM). Irradiated samples were centrifuged (1-1.5×1000 RPM, Sorvall RT6000B Refrigerated Centrifuge with Sorvall rotor H1000B) for approximately 3 minutes and then 50 μl of substrate solution were added. The samples with added substrate were incubated at 37° C. with shaking and absorbance at 406-620 mn determined at 20 minute intervals beginning 5 minutes after addition of substrate to the sample.

Results

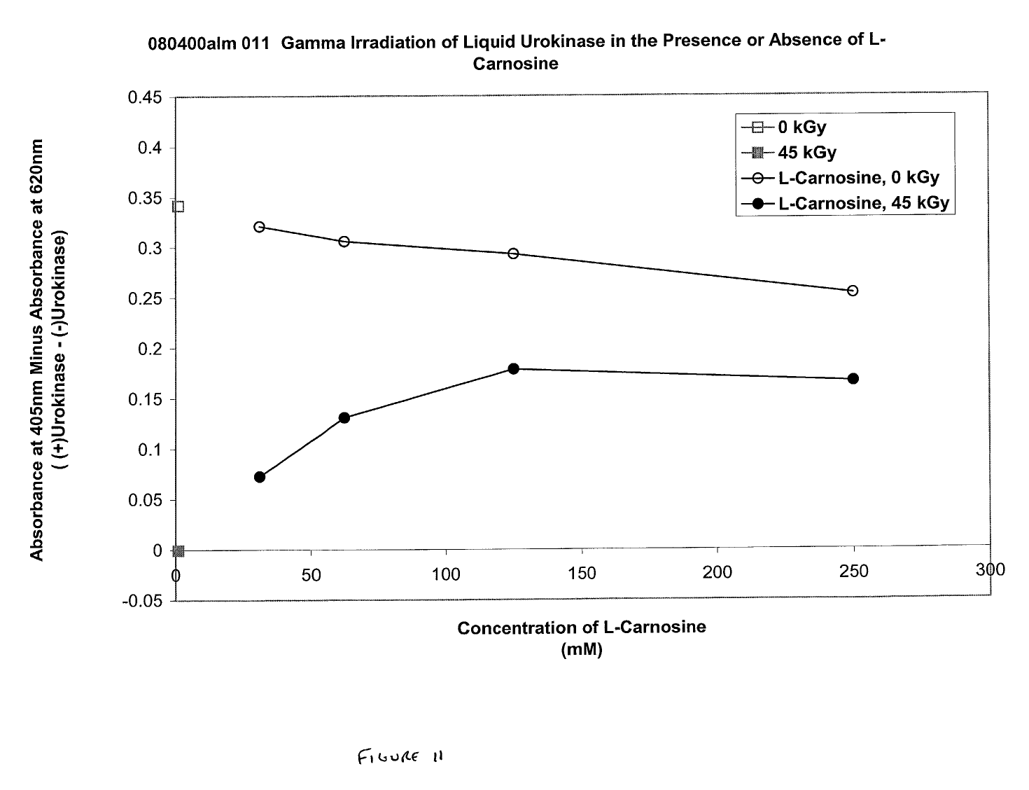

As shown in FIG. 11, L-camosine showed a concentration dependent protection of liquid urokinase irradiated to a total dose of 45 kGy. At concentrations of 125 and 250 mM, L-carnosine protected approximately 60-65% of the activity of irradiated liquid urokinase.

### Example 12

In this experiment, the protective effect of L-carnosine on gamma irradiated immobilized anti-insulin monoclonal immunoglobulin was evaluated.

Methods

L-camosine was prepared as a 100 mM solution in PBS pH 8-8.5. Approximately 100 μl of this solution was added to each sample being irradiated. Samples were irradiated at a dose rate of 1.92 kGy/hr to a total dose of 45 kGy at 4° C.

Monoclonal immunoglobulin activity was determined by a standard ELISA protocol. Maxisorp plates were coated with human recombinant insulin at 2 μg/ml overnight at 4° C. The plate was blocked with 200 μl of blocking buffer (PBS, pH 7.4, 2% BSA) for two hours at 37° C. and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed six times with wash buffers. One hundred pl of Sigma-104 substrate (1 mg/ml in DEA buffer) was added to each well and reacted at room temperature. The plate was read on a Multiskan MCC/340 at 405 nM with the 620 nM absorbance subtracted.

Results

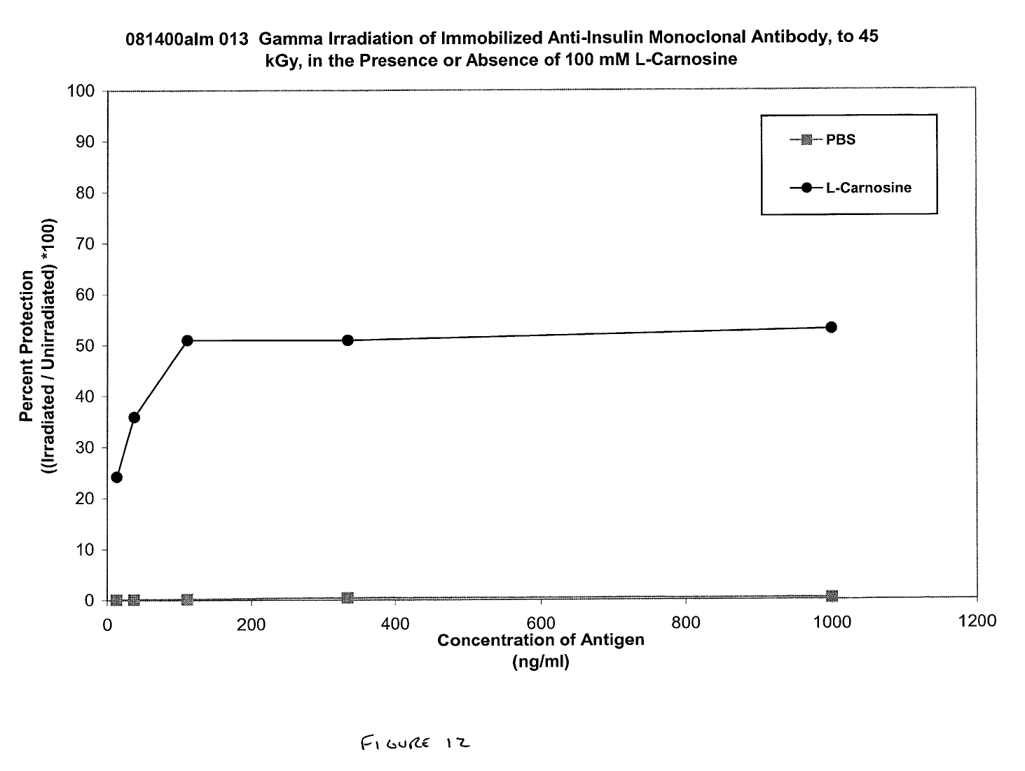

As shown in FIG. 12, samples of immobilized anti-insulin monoclonal immunoglobulin lost all binding activity when gamma irradiated to 45 kGy. In contrast, samples containing L-carnosine retained about 50% of binding activity following gamma irradiation to 45 kGy.

### Example 13

In this experiment, the protective effect of L-carnosine, alone or in combination with ascorbate, on gamma irradiated immobilized anti-insulin monoclonal immunoglobulin was evaluated.

Methods

L-carnosine was prepared as a solution in PBS pH 8-8.5 and added to each sample being irradiated across a range of concentration (25 mM, 50 mM, 100 mM or 200 mM). Ascorbate (either 50 mM or 200 mM) was added to some of the samples prior to irradiation. Samples were irradiated at a dose rate of 1.92 kGy/hr to a total dose of 45 kGy at 4° C.

Monoclonal immunoglobulin activity was determined by a standard ELISA protocol. Maxisorp plates were coated with human recombinant insulin at 2 μg/ml overnight at 4° C. The plate was blocked with 200 μl of blocking buffer (PBS, pH 7.4, 2% BSA) for two hours at 37° C. and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed six times with wash buffers. One hundred μl of Sigma-104 substrate (1 mg/ml in DEA buffer) was added to each well and reacted at room temperature. The plate was read on a Multiskan MCC/340 at 405 nM with the 620 nM absorbance subtracted.

Results

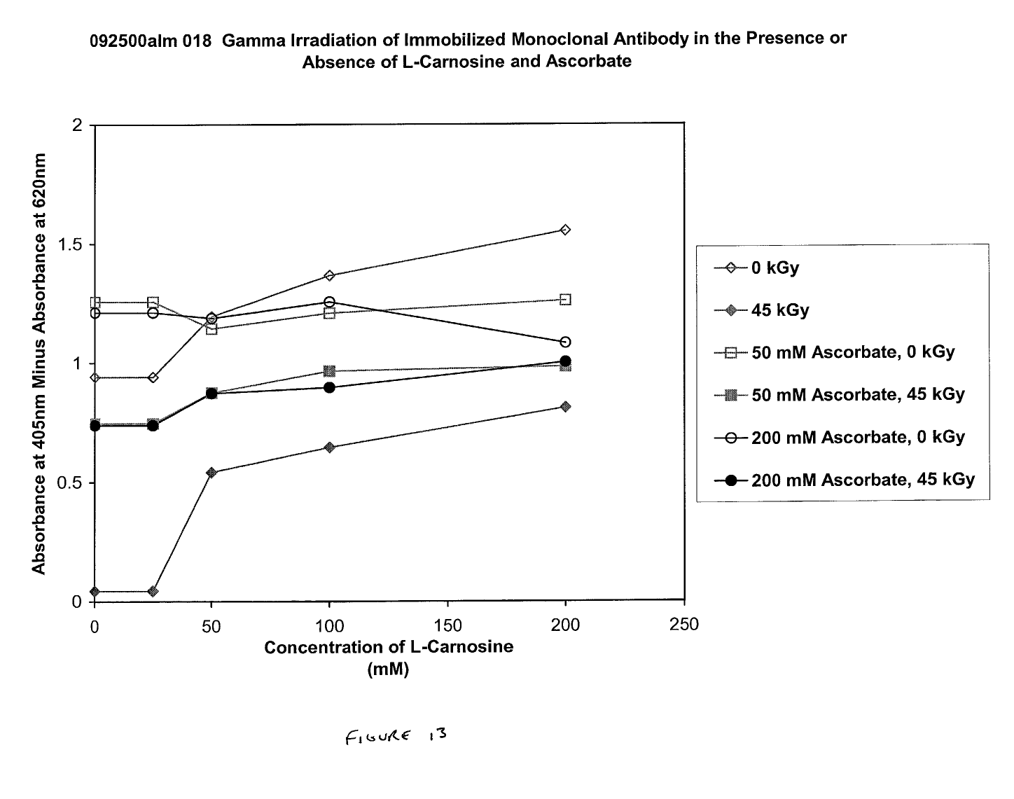

As shown in FIG. 13, samples of immobilized anti-insulin monoclonal immunoglobulin lost all binding activity when gamma irradiated to 45 kGy. In contrast, samples containing at least 50 mM L-carnosine retained about 50% of binding activity following gamma irradiation to 45 kGy. No additional protection was observed in the samples containing ascorbate as well, i.e. about 50% of binding activity was retained in samples containing at least 50 mM L-carnosine.

### Example 14

In this experiment, the protective effect of L-camosine, alone or in combination with scorbate, on gamma irradiated lyophilized Factor VIII was evaluated.

Methods

Samples containing Factor VIII and the stabilizer(s) of interest were lyophilized and stoppered under vacuum. Samples were irradiated at a dose rate of 1.9 kGy/hr to a total dose of 45 kGy at 4° C. Following irradiation, samples were reconstituted with water containing BSA (125 mg/ml) and Factor VIII activity was determined by a one-stage clotting assay using an MLA Electra 1400C Automatic Coagulation Analyzer.

Results

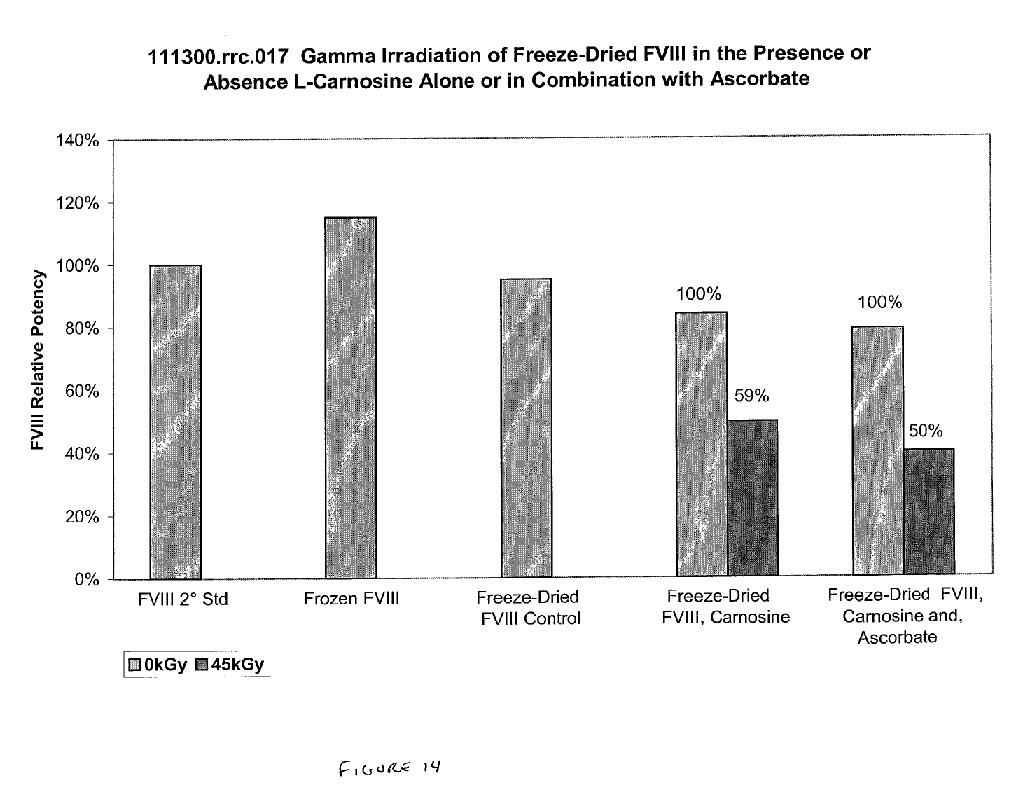

As shown in FIG. 14, L-camosine substantially improved the retention of Factor VIII clotting activity following gamma irradiation.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

What is claimed is:

1. A method for sterilizing a biological material that is sensitive to radiation, said method comprising:
(i) adding to said biological material at least one dipeptide stabilizer in an amount effective to protect said biological material from said radiation; and
(ii) irradiating said biological material with a suitable radiation at an effective rate for a time effective to sterilize said biological material.

2. A method for sterilizing a biological material that is sensitive to radiation, said method comprising:
(i) reducing the residual solvent content of said biological material;
(ii) adding to said biological material at least one dipeptide stabilizer; and
(iii) irradiating said biological material with a suitable radiation at an effective rate for a time effective to sterilize said biological material, wherein the level of said residual solvent content and the amount of said dipeptide stabilizer are together effective to protect said biological material from said radiation, and further wherein steps (i) and (ii) may be performed in inverse order.

3. A method for sterilizing a biological material that is sensitive to radiation, said method comprising:
(i) reducing the temperature of said biological material;
(ii) adding to said biological material at least one dipeptide stabilizer; and
(iii) irradiating said biological material with a suitable radiation at an effective rate for a time effective to sterilize said biological material, wherein the temperature and the amount of said dipeptide stabilizer are together effective to protect said biological material from said radiation, and further wherein steps (i) and (ii) may be performed in inverse order.

4. The method according to claim 2, wherein said solvent is water.

5. The method according to claim 4, wherein said residual water content is reduced by the addition of an organic solvent.

6. The method according to claim 2, wherein said solvent is an organic solvent.

7. The method according to claim 2, wherein said biological material is suspended in an organic solvent following reduction of said residual solvent content.

8. The method according to claim 1, 2 or 3, wherein said effective rate is not more than about 3.0 kGy/hour.

9. The method according to claim 1, 2 or 3, wherein said effective rate is not more than about 2.0 kGy/hr.

10. The method according to claim 1, 2 or 3, wherein said effective rate is not more than about 1.0 kGy/hr.

11. The method according to claim 1, 2 or 3, wherein said effective rate is not more than about 0.3 kGy/hr.

12. The method according to claim 1, 2 or 3, wherein said effective rate is more than about 3.0 kGy/hour.

13. The method according to claim 1, 2 or 3, wherein said effective rate is at least about 6.0 kGy/hour.

14. The method according to claim 1, 2 or 3, wherein said effective rate is at least about 18.0 kGy/hour.

15. The method according to claim 1, 2 or 3, wherein said effective rate is at least about 30.0 kGy/hour.

16. The method according to claim 1, 2 or 3, wherein said effective rate is at least about 45 kGy/hour.

17. The method according to claim 1, 2 or 3, wherein said biological material is maintained in a low oxygen atmosphere.

18. The method according to claim 1, 2 or 3, wherein said biological material is maintained in an atmosphere comprising at least one noble gas.

19. The method according to claim 18, wherein said noble gas is argon.

20. The method according to claim 1, 2 or 3, wherein said biological material is maintained in a vacuum.

21. The method according to claim 2, wherein said residual solvent content is reduced by a method selected from the group consisting of lyophilization, drying, concentration, addition of solute, evaporation, chemical extraction, spray-drying, and vitrification.

22. The method according to claim 2, wherein said residual solvent content is less than about 15%.

22. The method according to claim 2, wherein said residual solvent content is less than about 10%.

23. The method according to claim 2, wherein said residual solvent content is less than about 3%.

24. The method according to claim 2, wherein said residual solvent content is less than about 2%.

25. The method according to claim 2, wherein said residual solvent content is less than about 1%.

26. The method according to claim 2, wherein said residual solvent content is less than about 0.5%.

27. The method according to claim 2, wherein said residual solvent content is less than about 0.08%.

28. The method according to claim 1, 2 or 3, wherein at least one sensitizer is added to said biological material prior to said step of irradiating said biological material.

29. The method according to claim 1, 2 or 3, wherein said biological material contains at least one prion as a biological contaminant or pathogen.

30. The method according to claim 1, 2 or 3, wherein said biological material contains at least one biological contaminant or pathogen selected from the group consisting of viruses, bacteria and fungi.

29. The method according to claim 1, 2 or 3, wherein at least one additional stabilizer is added to said biological material prior to said step of irradiating said biological material.

30. The method according to claim 27, wherein said at least one additional stabilizer is an antioxidant.

31. The method according to claim 27, wherein said at least one additional stabilizer is a free radical scavenger.

32. The method according to claim 27, wherein said at least one additional stabilizer is a combination stabilizer.

33. The method according to claim 27, wherein said at least one additional stabilizer is a ligand.

34. The method according to claim 33, wherein said ligand is heparin.

35. The method according to claim 27, wherein said at least one additional stabilizer reduces damage due to reactive oxygen species.

36. The method according to claim 27, wherein said at least one additional stabilizer is selected from the group consisting of: ascorbic acid or a salt or ester thereof; glutathione; 6-hydroxy-2,5,7,8-tetramethylchroman-2-carboxylic acid; uric acid or a salt or ester thereof; methionine; histidine; N-acetyl cysteine; lipoic acid; sodium formaldehyde sulfoxylate; gallic acid or a derivative thereof; propyl gallate and mixtures of two or more thereof.

37. The method according to claim 36, wherein said mixtures of two or more additional stabilizers are selected from the group consisting of: mixtures of ascorbic acid, or a salt or ester thereof, and uric acid, or a salt or ester thereof; mixtures of ascorbic acid, or a salt or ester thereof, and 6-hydroxy-2,5,7,8-tetramethylchroman-2-carboxylic acid; mixtures of ascorbic acid, or a salt or ester thereof, uric acid, or a salt or ester thereof, and 6-hydroxy-2,5,7,8-tetramethylchroman-2-carboxylic acid; and mixtures of uric acid, or a salt or ester thereof; lipoic acid; sodium formaldehyde sulfoxylate; gallic acid or a derivative thereof; propyl gallate and 6-hydroxy-2,5,7,8-tetramethylchroman-2-carboxylic acid.

38. The method according to claim 1, 2 or 3, wherein said at least one dipeptide stabilizer is selected from the group consisting of glycol-glycine (Gly—Gly), carnosine and anserine.

39. The method according to claim 1, 2 or 3, wherein said radiation is corpuscular radiation or electromagnetic radiation, or a mixture thereof.

40. The method according to claim 39, wherein said electromagnetic radiation is selected from the group consisting of radio waves, microwaves, visible and invisible light, ultraviolet light, x-ray radiation, gamma radiation and combinations thereof.

41. The method according to claim 1, 2 or 3, wherein said radiation is gamma radiation.

42. The method according to claim 1, 2 or 3, wherein said radiation is e-beam radiation.

43. The method according to claim 1, 2 or 3, wherein said radiation is visible light.

44. The method according to claim 1, 2 or 3, wherein said radiation is ultraviolet light.

45. The method according to claim 1, 2 or 3, wherein said radiation is x-ray radiation.

46. The method according to claim 1, 2 or 3, wherein said radiation is polychromatic visible light.

47. The method according to claim 1, 2 or 3, wherein said radiation is infrared.

48. The method according to claim 1, 2 or 3, wherein said radiation is a combination of one or more wavelengths of visible and ultraviolet light.

49. The method according to claim 1, 2 or 3, wherein said irradiation is conducted at ambient temperature.

50. The method according to claim 1, 2 or 3, wherein said irradiation is conducted at a temperature below ambient temperature.

51. The method according to claim 1, 2 or 3, wherein said irradiation is conducted below the freezing point of said biological material.

52. The method according to claim 1, 2 or 3, wherein said irradiation is conducted below the eutectic point of said biological material.

53. The method according to claim 1, 2 or 3, wherein said irradiation is conducted at a temperature above ambient temperature.

54. A composition comprising at least one biological material and at least one dipeptide stabilizer in an amount effective to preserve said biological material for its intended use following sterilization with radiation.

55. The composition according to claim 54, further comprising at least one additional stabilizer selected from the group consisting of: ascorbic acid or a salt or ester thereof; glutathione; 6-hydroxy-2,5,7,8-tetramethylchroman-2-carboxylic acid; uric acid or a salt or ester thereof; methionine; histidine; N-acetyl cysteine; diosmin; silymarin; lipoic acid; sodium formaldehyde sulfoxylate; gallic acid or a derivative thereof; propyl gallate; a mixture of ascorbic acid, or a salt or ester thereof, and uric acid, or a salt or ester thereof; a mixture of ascorbic acid, or a salt or ester thereof, and 6-hydroxy-2,5,7,8-tetramethylchroman-2-carboxylic acid; a mixture of ascorbic acid, or a salt or ester thereof, uric acid, or a salt or ester thereof, and 6-hydroxy-2,5,7,8-tetramethylchroman-2-carboxylic acid; and a mixture of uric acid, or a salt or ester thereof and 6-hydroxy-2,5,7,8-tetramethylchroman-2-carboxylic acid, said at least one additional stabilizer is present in an amount effective to preserve said biological material for its intended use following sterilization with radiation.

56. The composition of claim 54, wherein the residual solvent content is sufficiently low to preserve said biological material, during sterilization by irradiation, for its intended use following sterilization with radiation.

57. The composition of claim 56, wherein said residual solvent content is less than about 15%.

58. The composition of claim 56, wherein said residual solvent content is less than about 10%.

59. The composition of claim 56, wherein said residual solvent content is less than about 5%.

60. The composition of claim 56, wherein said residual solvent content is less than about 2%.

61. The composition of claim 56, wherein said residual solvent content is less than about 1%.

62. The composition of claim 56, wherein said residual solvent content is less than about 0.5%.

63. The composition of claim 56, wherein said residual solvent content is less than about 0.08%.

64. The composition of claim 56, wherein said biological material is glassy or vitrified.

65. The composition of claim 54, wherein said biological material is selected from the group consisting of monoclonal immunoglobulins, polyclonal immunoglobulins, glycosidases, sulfatases, urokinase and Factor VIII.

66. The composition of claim 56, wherein the concentration of said biological material is at least about 0.5%.

67. The composition of claim 56, wherein the concentration of said biological material is at least about 1%.

68. The composition of claim 56, wherein the concentration of said biological material is at least about 5%.

69. The composition of claim 56, wherein the concentration of said biological material is at least about 10%.

70. The composition of claim 56, wherein the concentration of said biological material is at least about 15%.

71. The composition of claim 56, wherein the concentration of said biological material is at least about 20%.

72. The composition of claim 56, wherein the concentration of said biological material is at least about 25%.

73. The composition of claim 56, wherein the concentration of said biological material is at least about 50%.
